(19) United States
(12) Patent Application Publication
(10) Pub. No.: US 2008/0261986 A1
(43) Pub. Date: Oct. 23, 2008

(54) PHARMACEUTICAL FORMULATIONS FOR IONTOPHORETIC DELIVERY OF AN ANTI-FUNGAL DRUG

(76) Inventors: Phillip M. Friden, Bedford, MA (US); Hyun Kim, Weston, MA (US); Bireswar Chakraborty, Andover, MA (US)

Correspondence Address: ELMORE PATENT LAW GROUP, PC, 515 Groton Road, Unit 1R, Westford, MA, 01886, US

(21) Appl. No.: 12/056,802

(22) Filed: Mar. 27, 2008

Related U.S. Application Data
(60) Provisional application No. 60/921,170, filed on Mar. 30, 2007.
(60) Provisional application No. 61/014,592, filed on Dec. 18, 2007.

(51) Int. Cl. A61K 31/135; A61P 17/00; A61P 31/10; A61K 31/496; A61K 31/4164; A61K 31/4412; A61K 31/4196; A61K 31/5375

(52) U.S. Cl. 514/239.5; 514/655; 514/254.07; 514/399; 514/345; 514/383

(57) ABSTRACT

Pharmaceutical formulations suitable for iontophoresis thereof that provide enhanced iontophoretic delivery of an anti-fungal drug to at least one body surface are described. Also described are pharmaceutical formulations suitable for iontophoresis comprising terbinafine and methods for administering terbinafine to a body surface via iontophoresis. In one embodiment, the body surface includes a nail plate and/or the skin.

### RELATED APPLICATION

This application claims the benefit of Provisional Application No. 60/921,170 filed on Mar. 30, 2007 and Provisional Application No. 61/014,592 filed on Dec. 18, 2007. The entire teachings of the above applications are incorporated herein by reference.

### BACKGROUND OF THE INVENTION

An iontophoretic delivery system is, for example, a drug delivery system that releases drug at a controlled rate to the target tissue upon application. The advantages of systems wherein drug is delivered locally via iontophoresis are the ease of use, relatively safe administration, the ability to finely modulate the dose by changing the time of application and/or the current level and the ability to interrupt administration by simply stopping the current and/or peeling off or removing it from the skin or other body surface whenever an overdosing is suspected. The total skin surface area of an adult is about 2 m². In recent years iontophoretic delivery of drugs has attracted wide attention as a better way of administering drugs for local as well as systemic effects. The design of iontophoretic delivery systems can usually be such that the side effects generally seen with the systemic administration of conventional dosage forms are minimized.

Iontophoresis has been employed for many years as a means for applying medication locally through a patient's skin and for delivering medicaments to the eyes and ears. The application of an electric field to the skin is known to greatly enhance the ability of the drugs to penetrate the target tissue. The use of iontophoretic transdermal delivery techniques has obviated the need for hypodermic injection for some medicaments, thereby eliminating the concomitant problems of trauma, pain and risk of infection to the patient.

Iontophoresis involves the application of an electromotive force to drive or repel ions through the dermal layers into a target tissue. Particularly suitable target tissues include those adjacent to the delivery site for localized treatment. Uncharged molecules can also be delivered using iontophoresis via a process called electroosmosis.

Regardless of the charge of the medicament to be administered, an iontophoretic delivery device employs two electrodes (an anode and a cathode) in conjunction with the patient's skin to form a closed circuit between one of the electrodes (referred to herein alternatively as a “working” or “application” or “applicator” electrode) which is positioned at the site of drug delivery and a passive or “grounding” electrode affixed to a second site on the skin to enhance the rate of penetration of the medicament into the skin adjacent to the applicator electrode.

U.S. Pat. No. 6,477,410 issued to Henley et al. describes the use of iontophoresis for drug delivery. However, there remains a need for improved formulations that facilitate the delivery of specific active agents. Such active agents include anti-fungal agents. Among the fungal infections that may be treated with anti-fungal drugs are fungal infections of the nail and/or the skin. These fungal infections occur under the nail and in the cuticle and as such, are often not amenable to topical treatments as these may not effectively penetrate the area under the nail. Lacquers with penetration enhancer may be used for such topical treatments, but delivery to the affected area remains poor in many cases. For this reason, certain fungal infections of the nail are treated with oral formulations of anti-fungal drugs. Oral administration of anti-fungals, however, may be associated with adverse effects. For example, oral administration of the anti-fungal drug, terbinafine hydrochloride, is associated with hepatotoxicity, gastrointestinal symptoms (including diarrhea, dyspepsia, and abdominal pain), rashes, urticaria and pruritus. It would therefore be advantageous to develop improved formulations of anti-fungal drugs and methods of administering anti-fungal drugs that result in increased permeation of topically administered anti-fungal drug into the nail.

### SUMMARY OF THE INVENTION

The present invention provides pharmaceutical formulations suitable for iontophoresis that provide enhanced iontophoretic delivery of an anti-fungal drug to at least one body surface. In another embodiment, the invention is directed to a method for administering an anti-fungal drug to a patient in need thereof comprising iontophoretically administering to a body surface of the patient a formulation comprising terbinafine hydrochloride. In yet another embodiment, the invention is directed to a method of treating a fungal infection comprising iontophoretically administering to a body surface of the patient a formulation comprising an antifungal drug.

Other embodiments of the present invention are directed to pharmaceutical formulations suitable for iontophoresis that provide enhanced iontophoretic delivery of terbinafine to at least one body surface. In one embodiment, the invention is directed to a method for administering terbinafine to a patient in need thereof comprising iontophoretically administering to a body surface of the patient a formulation comprising terbinafine hydrochloride. In another embodiment, the invention is directed to a method of treating a fungal infection comprising iontophoretically administering to a body surface of the patient a formulation comprising terbinafine. In a further embodiment, the terbinafine is terbinafine hydrochloride.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a graph comparing the cumulative amount of terbinafine hydrochloride (μg/cm²) that permeated the hoof membrane over 24 hours following 1 hour of iontophoresis (1.0 mA/cm² current density) to passive delivery.

FIG. 2 is a graph showing the effect of the type of electrode on the amount of cumulative drug (μg/ml) that permeated the hoof over time.

FIG. 3A is a bar graph showing the amount of terbinafine hydrochloride (10 mg/mL in water and Tween-80) that permeated through the nail (μg/cm²) after 0.5 mA/cm² for 1 hr.

FIG. 3B is a bar graph showing the amount of terbinafine hydrochloride (10 mg/mL in water and Tween-80) that loaded in the nail (mg drug per g nail) after 0.5 mA/cm² for 1 hr.

FIG. 4 is a bar graph showing the effect of current duration on permeation (μg/cm²) of terbinafine hydrochloride (10 mg/mL in water and Tween-80) through the nail using 0.5 mA/cm² current density.

FIG. 5A is a bar graph comparing the amount of drug (μg/cm²) that permeated the hoof membrane for four different formulations of terbinafine HCl (TF-1, TF-5, TF-10 and TF-11 described in Example 8 below), a formulation of terbinafine in water and Tween-80 and LAMISIL® after iontophoresis.

FIG. 5B is a bar graph comparing the amount of drug (mg/g) in the hoof for four different formulations of terbinafine HCl, a formulation of terbinafine in water and Tween-80 and LAMISIL® after iontophoresis.

FIG. 6A is a bar graph comparing the amount of drug (μg/cm²) that permeated the hoof for different formulations of terbinafine hydrochloride after iontophoresis (described in Example 9 below).

FIG. 6B is a bar graph comparing the amount of drug (mg/g) loaded in the hoof for different formulations of terbinafine hydrochloride after iontophoresis.

FIG. 7A is a bar graph comparing the amount of drug (μg/cm²) that permeated the nail for different formulations of terbinafine hydrochloride after iontophoretic and passive delivery (described in Example 10 below).

FIG. 7B is a bar graph comparing the amount of drug loaded (mg/g) in the nail for different formulations of terbinafine hydrochloride after iontophoretic and passive delivery.

FIG. 7C is a bar graph comparing the amount of drug that permeated and loaded in the nail (ug) for different formulation of terbinafine hydrochloride after iontophoretic delivery.

FIG. 8A is a bar graph comparing the amount of drug (mcg/cm²) that permeated the nail after 0.1, 0.25, 0.5, 0.75 or 1 mA/cm² was applied for 15, 30, 45 and 60 minutes.

FIG. 8B is a bar graph comparing the amount of drug (mg/g) loaded in the nail after 0.25, 0.5, 0.75 and 1 mA/cm² was applied for 30 or 60 minutes.

FIG. 8C is a plot showing the amount of drug (μg/cm²) that permeated the nail after iontophoretic delivery at various Coulombic doses (mA*min) in the Franz diffusion cell.

FIG. 8D is a plot showing the amount of drug (mg/g) loaded into the nail after iontophoretic delivery at various Coulombic doses (mA*min) in the Franz diffusion cell.

FIG. 9A is drawing depicting a cross-sectional view of the nail on agarose model.

FIG. 9B is a bar graph showing the amount of drug (μg/cm²) that permeated the nail after iontophoretic and passive delivery using a nail-only applicator.

FIG. 9C is a bar graph showing the amount of drug (mg/g) loaded into the nail after iontophoretic and passive delivery using a nail-only applicator.

FIG. 9D is a bar graph showing the amount of drug (μg/cm²) that permeated the nail after iontophoretic and passive delivery using a nail and skin applicator.

FIG. 9E is a bar graph showing the amount of drug (mg/g) loaded in the nail after iontophoretic and passive delivery using a nail and skin applicator.

FIG. 9F is a plot showing the amount of drug (mg/g) loaded in the nail after iontophoretic delivery at various Coulombic doses (mA*min) using a nail only and nail and skin applicator.

FIG. 10 is a bar graph showing the amount of drug (μg/cm²) delivered to the stratum corneum, the skin and receptor fluid using the in vitro hairless rat skin model.

FIG. 11 is a plot showing the amount of drug released (μg) from nails loaded iontophoretically or passively.

FIGS. 12A and B are pictures showing zones of inhibition on agar plates inoculated with T. rubrum treated with nail pieces loaded with terbinafine hydrochloride passively or iontophoretically.

FIG. 12C is a bar graph showing the initial amount of drug (μg) in nail samples after iontophoretic or passive delivery.

FIG. 12D is a bar graph showing the amount of drug (μg) released from nail samples after 4 days as calculated from the zones of inhibition observed on agar plates inoculated with T. rubrum.

FIG. 12E is a bar graph showing the amount of drug (μg) released from nail samples for up to 36 days after drug was loaded into the nail passively or iontophoretically.

FIG. 12F is a plot showing the cumulative amount of drug (μg) released from nail samples for up to 36 days after drug was loaded into the nail passively or iontophoretically.

### DETAILED DESCRIPTION OF THE INVENTION

The invention is directed to pharmaceutical formulations of an anti-fungal drug that are suitable for iontophoresis, methods of administering an anti-fungal drug to an affected body surface of a patient in need thereof and methods of treating a fungal infection in a patient suffering therefrom comprising iontophoretically administering an anti-fungal drug to an affected body surface. The invention is also directed to pharmaceutical formulations of terbinafine that are suitable for iontophoresis, methods of administering terbinafine to an affected body surface of a patient in need thereof and methods of treating a fungal infection in a patient suffering therefrom comprising iontophoretically administering terbinafine to an affected body surface.

As used herein, the words “a” and “an” are each meant to include one or more unless otherwise specified.

As used herein, an antifungal drug is a drug that directly or indirectly inhibits or reduces fungal growth, contamination or infection, or a symptom or condition associated with fungal growth, contamination or infection, or that decreases or reduces recurrence of or susceptibility to fungal growth, contamination or infection by a fungus, regardless of the mode of action. Classes of antifungal drugs include, but are not limited to, allylamines, azoles, pyrimidines, tetraenes, thiocarbamates, sulfonamides, glucan synthesis inhibitors. Allylamines include, for example, amorolfine, butenafine, naftifine and terbinafine. Azoles include, for example, ketoconazole, fluconazole, elubiol, econazole, econaxole, itraconazole, isoconazole, imidazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, flutrimazole. Polyenes include, for example, nystatin, pimaricin and amphotericin B. Pyrimidines include, for example, flucytosine. Tetraenes include, for example, natamycin. Thiocarbamates include, for example, tolnaftate. Sulfonamides include, for example, mafenidine. Other antifungal drugs include ciclopirox and ciclopirox olamine.

In one embodiment, the antifungal drug is an allylamine. In another embodiment, the antifungal drug is an azole.

In another embodiment, the antifungal drug is selected from the group consisting of terbinafine, ketoconazole, econazole, ciclopirox, ciclopirox olamine, fluconazole, itraconazole and amorolfine. In yet another embodiment, the antifungal agent is selected from the group consisting of terbinafine, ciclopirox and ciclopirox olamine. In an additional embodiment, the antifungal agent is selected from the group consisting of terbinafine and ciclopirox olamine. In certain embodiments, the antifungal agent is terbinafine. In a further embodiment, the terbinafine is terbinafine hydrochloride.

Terbinafine hydrochloride is a synthetic allylamine derivative and has the chemical name (E)-N-(6,6-dimethyl-2-hepten-4-ynyl)-N-methyl-1-naphthylmethamine hydrochloride. Terbinafine hydrochloride is currently marketed under the trade name Lamisil® and is sold both as a tablet and as cream and solution formulations. Terbinafine hydrochloride has been shown to be effective in the treatment of fungal infections by inhibiting the enzyme squalene epoxidase which inhibits the biosynthesis of ergosterol. Ergosterol is a necessary component of the fungal cell membrane and inhibition of its biosynthesis leads to fungal cell death. Terbinafine hydrochloride is mainly effective against the dermatophyte group of fungi, including, for example, Trichophyton rubrum, Trichophyton mentagrophytes and Epidermophytum floccosum.

In one embodiment, the invention is directed to a formulation suitable for iontophoresis comprising an aqueous composition of an antifungal drug, a pharmaceutically acceptable excipient or carrier and a non-ionic surfactant. In another embodiment, the invention is directed to a formulation comprising an aqueous composition of terbinafine, a pharmaceutically acceptable excipient or carrier and a non-ionic surfactant. In an additional embodiment, the formulation may contain counter ions that can make ion pairs with the ionized drug and improve delivery of the ionized drug. In another embodiment the invention is directed to a formulation suitable for iontophoresis comprising an antifungal drug and one or more solvents and co-solvents. In yet another embodiment, the invention is directed to a formulation suitable for iontophoresis comprising an antifungal drug and a permeation enhancer. In a further embodiment, the invention is directed to a formulation suitable for iontophoresis comprising an antifungal drug, one or more solvents or co-solvents and a permeation enhancer. In an additional embodiment, the invention is directed to a formulation suitable for iontophoresis comprising an antifungal drug, one or more solvents or co-solvents, a permeation enhancer and a non-ionic surfactant.

In yet another embodiment, the formulation can comprise an anti-fungal drug and one or more chemical permeation or penetration enhancers. A “permeation enhancer” or “penetration enhancer” is a material which achieves permeation enhancement or an increase in the permeability of the skin and/or nail to a pharmacologically active agent. The terms “permeation enhancer” and “penetration enhancer” are used interchangeably herein. Examples of such permeation enhancers include, but are not limited to, N-acetylcysteine, urea, salicylic acid, linoleic acid, benzoic acid, oleic acid, cysteine hydrochloride, cyclodextrin, dimethyl sulfoxide, polyethylene glycol (PEG), polyvinylpyrolidone (PVP), dimyristoyl phosphatidylserine, saturated fatty acids (including, for example, stearic acid and palmitic acid) and combinations thereof. In one embodiment, the formulation comprises a permeation enhancer that is a keratolytic agent selected from the group consisting of benzoic acid, oleic acid, cysteine hydrochloride, N-acetylcysteine and urea. In an additional embodiment, the formulation comprises a permeation enhancer selected from the group consisting of polyethylene glycol and polyvinylpyrrolidone. In another embodiment, the formulation comprises a penetration enhancer selected from the group consisting of benzoic acid, polyethylene glycol, polyvinylpyrrolidone and combinations thereof. In certain embodiments, the permeation enhancer utilized is a permeation enhancer that enhances permeation into and through the nail to the nail bed or into the skin. The amount of permeation enhancer that is utilized is the amount that increases the permeation of terbinafine compared to permeation of terbinafine in the absence of the permeation enhancer. In one embodiment, the one or more permeation enhancers are each included in the composition in an amount from about 0.01 to about 50% (w/w).

In another embodiment, the formulation comprises a permeation enhancer selected from the group consisting of benzoic acid, oleic acid, salicylic acid, cysteine hydrochloride, N-acetylcysteine and urea wherein the permeation enhancer is included in an amount from about 0.05 to about 5.0% (w/w). In yet another embodiment, the formulation comprises a permeation enhancer selected from the group consisting of benzoic acid, oleic acid, salicylic acid, cysteine hydrochloride, N-acetylcysteine and urea wherein the permeation enhancer is included in an amount from about 0.05 to about 2.0% (w/w). In a further embodiment, the formulation comprises a permeation enhancer selected from the group consisting of benzoic acid, oleic acid, salicylic acid, cysteine hydrochloride, N-acetylcysteine and urea wherein the permeation enhancer is included in an amount from about 0.05 to about 1.0% (w/w). In yet another embodiment, the formulation comprises a permeation enhancer selected from the group consisting of benzoic acid, oleic acid, salicylic acid, cysteine hydrochloride, N-acetylcysteine and urea wherein the permeation enhancer is included in the composition in an amount from about 0.01 to about 5.0% relative to the amount of terbinafine in the composition.

In another embodiment, the formulation comprises polyethylene glycol. Polyethylene glycol may act as a permeation enhancer and/or a solvent or co-solvent as described below. As will be appreciated by one having skill in the art, polyethylene glycol of various molecular weights can be used in the inventive formulation, including, but not limited to polyethylene glycol (PEG) 200, PEG 400, PEG 600, PEG 1000 and PEG 3550. In another embodiment, the polyethylene glycol is PEG 400. In an additional embodiment, the formulation comprises polyethylene glycol wherein the polyethylene glycol is included in the formulation in an amount from about 10 to about 50% (w/w). In yet another embodiment, the formulation comprises polyethylene glycol wherein polyethylene glycol is included in the formulation in an amount from about 20 to about 50% (w/w). In a further embodiment, the formulation comprises polyethylene glycol and is included in the formulation from about 25 to about 45% (w/w). In an additional embodiment, the formulation comprises polyethylene glycol and polyethylene glycol is included in the formulation in amount of about 30% (w/w). In a further embodiment, the formulation comprises polyethylene glycol in an amount of about 40% (w/w).

In an additional embodiment, the formulation comprises polyvinylpyrrolidone (PVP). Polyvinylpyrrolidone may act as a permeation enhancer and/or a solvent or co-solvent as described below. As will be appreciated by one having ordinary skill in the art, polyvinylpyrrolidone of various molecular weights can be used in the inventive formulation. In one embodiment, the polyvinylpyrrolidone has a molecular weight from about 1000 to about 50,000. In another embodiment, the formulation comprises PVP and is included in the formulation in an amount from about 0.01 to about 10% (w/w). In yet another embodiment, the formulation comprises PVP and is included in the formulation in an amount from about 0.01 to about 5% (w/w). In a further embodiment, the formulation comprises PVP and is included in the formulation in an amount from about 1 to about 5% (w/w).

In another embodiment, the formulation comprises an antifungal drug and one or more solvents or co-solvents are selected from the group consisting of glycerin, an alcohol, glycol, a glycol monoether, a glycol diether, dimethylsulfoxide, caprolactam, dimethylisosorbide, isopropylidene glycerol, dimethylimidazolidinone, N-methyl-pyrrolidone-2, pyrrolidone-2, ethyl lactate, polyoxyethylenated C8-C10 glycerides, glyceryl laurate, dimethylacetamide, polyethylene glycol, polyvinylpyrrolidone and the like as well as combinations thereof. In yet another embodiment, the formulation comprises glycerin. In yet another embodiment, the formulation comprises an alcohol. Exemplary alcohols include, but are not limited to, ethanol, butanol, tert-butyl alcohol and benzyl alcohol. In one embodiment, the alcohol is ethanol. In a further embodiment, the formulation comprises glycerin and an alcohol. In an additional embodiment, the formulation comprises glycerin and ethanol.

The formulation can also contain a stabilizer such as an antioxidant or a chelating agent (including, for example, EDTA, disodium EDTA, butylated hydroxyl toluene, butylated hydroxy anisole, TPGS, sodium sulfites, ascorbic acid, vitamin E, etc.) and/or an alcohol (including, for example, ethyl alcohol). In another embodiment, the formulation may contain a preservative including, but not limited to, sodium benzoate, benzalkonium chloride, parabens (including methyl and propyl paraben), and the like. In a further embodiment, the formulation may contain an agent that affects protein binding including, but not limited to, linolenic acid, oleic acid, dimyristoyl phosphatidyl glycerol (DMPG), dimyristoyl phosphatidyl choline (DMPC) and isopropyl myristate.

As used herein, the term “pharmaceutically acceptable carrier or excipient” means any non-toxic diluent or other formulation auxiliary that is suitable for use in iontophoresis. Examples of pharmaceutically acceptable carriers or excipients include but are not limited to a solvent, co-solvents (including, for example, a solvent or co-solvent described above including, but not limited to, glycerin and/or an alcohol), a solubilizing agent (such as sorbitol or glycerin), a buffer, a pharmaceutically acceptable base, an alcohol such as benzyl alcohol and butanol and a viscosity modulating agent such as cellulose and its derivatives. The aqueous composition of terbinafine can comprise one or more solvents or co-solvents. In one embodiment, the solvent is water or a buffer. Buffers include, for example, phosphate buffer, citrate buffer, acetate buffer, piperazine-N,N′-bis(2-ethanesulfonic acid) (PIPES) buffer, dimethyl arsenate (Cacodylate) buffer and 2-(N-morpholino)ethanesulfonic acid (MES) buffer. In one embodiment, the buffer is a phosphate buffer. In another embodiment, the buffer is phosphate buffered saline.

In an additional embodiment, the antifungal agent is terbinafine and the pH of the formulation is less than about pH 7. In a further embodiment, the pH of the formulation is less than about 6. In an additional embodiment, the pH is less than about 5. In a further embodiment, the pH of the formulation is about 5. In another embodiment, the pH of the formulation is about 2.5 to about. 4.5. In a further embodiment, the pH of the formulation is about 3.

In another embodiment, the formulation is adsorbed into a foam material or used to swell a hydrogel.

In one embodiment, the viscosity of the formulation comprising terbinafine is similar to that of water. In another embodiment, the formulation is a liquid formulation.

Non-ionic surfactants can be included in the inventive formulation and include, for example, an ethoxylated polysorbate, polyethylene glycol, ethylene oxide/propylene oxide copolymer, and polyethoxylated castor oil. Ethoxylated polysorbates, include, for example, polysorbate-20 (Tween-20), polysorbate-40 (Tween-40) and polysorbate-80 (Tween-80). In another embodiment, the non-ionic surfactant is selected from the group consisting of polysorbate-20, polysorbate-40 and polysorbate-80. In one embodiment, the non-ionic surfactant comprises from about 2 to about 10% by weight of the solvent. In a further embodiment, the formulation comprises water and a non-ionic surfactant wherein the non-ionic surfactant comprises about 5% by weight of the solvent. In another embodiment, the formulation comprises water and a non-ionic surfactant wherein the non-ionic surfactant comprises about 5% by weight of the formulation. Polyethoxylated castor oils include, for example, Cremophors, such as polyethoxylated castor oil 35 (Cremophor RH40).

In one embodiment, the formulation comprises an anti-fungal drug in a therapeutically effective amount. A “therapeutically effective amount” is an amount of anti-fungal drug that is sufficient to prevent development of or alleviate to some extent one or more of a patient's symptoms of the disease being treated. In another embodiment, the formulation comprises an anti-fungal drug at a concentration sufficient to treat a fungal infection. In another embodiment, the anti-fungal drug is present at a concentration sufficient to treat a fungal infection of the nail. In yet another embodiment, the anti-fungal drug is present at a concentration sufficient to treat onychomycosis. In one embodiment, the antifungal drug is present at a concentration of at least about 5 mg/ml. In yet another embodiment, the antifungal drug is present at a concentration of at least about 10 mg/ml. In a further embodiment, the antifungal drug is present at a concentration of at least about 20 mg/ml.

In one embodiment, the formulation comprises terbinafine and the terbinafine is present in the formulation at a concentration sufficient to treat a fungal infection. In another embodiment, the terbinafine is present at a concentration sufficient to treat a fungal infection of the nail. In yet another embodiment, the terbinafine is present at a concentration sufficient to treat onychomycosis. In one embodiment, the terbinafine is present at a concentration of at least about 5 mg/ml. In another embodiment, the terbinafine is present at a concentration of at least about 10 mg/ml. In a further embodiment, the terbinafine is present at a concentration of at least about 25 mg/ml. In one embodiment, the terbinafine is present at a concentration from about 5 to about 100 mg/ml. In yet another embodiment, the terbinafine is present at a concentration from about 5 to about 50 mg/ml. In an additional embodiment, the terbinafine is present at a concentration from about 20 to about 50 mg/ml. In a further embodiment, the terbinafine is present at a concentration of about 40 mg/ml.

In another embodiment, the terbinafine is present at a concentration from about 5 to about 25 mg/ml. In another embodiment, the terbinafine is present at a concentration from about 5 to about 15 mg/ml. In yet another embodiment, the terbinafine is present at a concentration from about 5 to about 12 mg/ml. In a further embodiment, the terbinafine is present at a concentration from about 5 mg/ml to at least about 10 mg/ml. In yet another embodiment, the formulation comprises a salt. In a further embodiment, the formulation comprises a phosphate buffered saline.

In an additional embodiment, the terbinafine is present in the composition in an amount between about 1 and about 10% (w/w). In another embodiment, the terbinafine is present in the composition in an amount between about 2 and about 6% (w/w). In yet another embodiment, the terbinafine is present in the composition in an amount between about 6 and about 10% (w/w). In a further embodiment, the terbinafine is present in an amount of about 4% (w/w).

In one embodiment, the formulation has a viscosity from about 100 to about 1000 cp at 25° C. In a further embodiment, the formulation of the invention comprises a viscosity modifying agent. A viscosity modifying agent can be added to the formulation to achieve the desired viscosity. A viscosity modifying agent includes any agent that is capable of modulating the viscosity of a gel. Viscosity modifying agents useful in the practice of the invention include but are not limited to, ionic and non-ionic, high viscosity, water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose (HEC), hydroxypropyl methylcellulose, hydroxypropyl methylcellulose phthalate, methyl cellulose, carboxymethyl cellulose, and etherified cellulose; gums such as tragacanth and xanthan gum; sodium alginate, calcium alginate; gelatin, hyaluronic acid and salts thereof, chitosans, gellans or any combination thereof. It is preferred that non-basic viscosity enhancing agents, such as a neutral or acidic agent (such as, phosphoric acid or hydrochloric acid) be employed in order to facilitate achieving the desired pH of the formulation. If a uniform gel is desired, dispersing agents such as alcohol, sorbitol or glycerin can be added, or the gelling agent can be dispersed by trituration, mechanical mixing, or stirring, or combinations thereof. In one embodiment, the viscosity enhancing agent can also provide the acid, discussed above.

In one embodiment, the viscosity modifying agent is cellulose that has been modified such as by etherification or esterification. One such etherified cellulose polymer is sold under the trademark Natrosol® (Hercules-Aqualon, Wilmington, Del.). The pharmaceutically acceptable carrier or excipient may comprise about 0.1 to 10 weight percent of a viscosity modulating agent. The formulation of terbinafine hydrochloride can be a gel.

In certain embodiments of the invention, the formulation comprises terbinafine and a penetration enhancer. In a further embodiment, the formulation comprises terbinafine hydrochloride in an amount from about 1 to about 10% (w/w) and one or more additional components listed in Table A below in the following amounts:

TABLE A

| Component | Composition (%)(w/w) |
|---|---|
| Solvent or co-solvents | About 10 to about 50 |
| Surfactant/Solubilizer | About 1 to about 5 |
| Chelating agent | About 0.01 to about 0.10 |
| Antioxidant | About 0.005 to about 0.10 |
| Permeation enhancer | About 0.05 to about 50 |
| Preservative | About 0.01 to about 0.10 |
| Thickener, viscosity modulating agent | About 0.10 to about 0.50 |
| pH adjusting agent | As needed to adjust the pH to a |
| | pH from about 2.5 to about 4.5 |

It is to be understood that the formulation can comprise one or more of the components described above. In addition, it is to be understood that the formulation can comprise one or more of each type of component described above; for example, the formulation may comprise one or more solvent or co-solvents and/or one or more permeation enhancers.

In yet another embodiment, the formulation comprises terbinafine hydrochloride, one or more solvents or co-solvents and one or more permeation enhancers wherein the one or more solvents or co-solvents and permeation enhancers are present in an amount shown in Table A and wherein the terbinafine hydrochloride is present at a concentration from about 1 to about 10% (w/w). In one embodiment, the one or more solvents or co-solvents are selected from the group consisting of water, an alcohol and glycerin. An exemplary alcohol is ethanol. In yet another embodiment, the formulation comprises one or more permeation enhancers selected from the group consisting of benzoic acid, polyethylene glycol and polyvinylpyrrolidone. In an additional embodiment, the formulation comprises benzoic acid. In a further embodiment, the formulation comprises benzoic acid and a polyethylene glycol. In an additional embodiment, the formulation comprises benzoic acid and polyethylene glycol 400 (PEG 400).

In an additional embodiment, the formulation comprises terbinafine hydrochloride in an amount from about 1 to about 10% (w/w) and one or more of additional components listed in Table B below in the following amounts:

TABLE B

| Component | Composition (%)(w/w) |
|---|---|
| Glycerin, propylene glycol, polyethylene | About 10 to about 50 |
| glycol | |
| Polyvinylpyrrolidone | About 0.05 to about 10 |
| Ethyl alcohol or other alcohol | About 10 to about 50 |
| Polysorbate-80 | About 1 to about 5 |
| Water | About 25 to about 45 |
| Benzoic acid, oleic acid, cysteine | About 0.05 to about 2.0 |
| hydrochloride, salicylic acid, N-acetyl | |
| cysteine or urea | |
| Sodium benzoate, methyl paraben or | About 0.01 to about 0.10 |
| propyl paraben | |
| Hydroxyethyl cellulose | About 0.10 to about 0.50 |
| Phosphoric acid or hydrochloric acid | As needed to adjust |
| | the pH to a pH from |
| | about 2.5 to about 4.5 |

In a further embodiment, the formulation comprises terbinafine hydrochloride in an amount from about 1 to about 10% (w/w) and one or more of additional components listed in Table C below in the following amounts:

TABLE C

| Component | Composition (%)(w/w) |
|---|---|
| Glycerin | About 10 to about 50 |
| Ethyl alcohol | About 10 to about 50 |
| Polysorbate-80 | About 1 to about 5 |
| Water | About 25 to about 45 |
| Benzoic acid | About 0.05 to about 2.0 |
| Sodium benzoate, methyl paraben, propyl | About 0.01 to about 0.10 |
| paraben or butylated hydroxytoluene | |
| (BHT) | |
| Hydroxyethyl cellulose (HEC) | About 0.1 to about 0.5 |
| Phosphoric acid or hydrochloric acid | As needed to adjust |
| | the pH to a pH from |
| | about 2.5 to about 4.5 |

In one embodiment, the composition comprises terbinafine hydrochloride, ethanol, glycerin, water and polysorbate-80 wherein the ethanol, glycerin, water and polysorbate-80 are present in an amount encompassed by Table C.

In yet another embodiment, the formulation comprises between about 1 to about 6% (w/w) terbinafine hydrochloride and one or more additional components listed in Table D below in the following amounts:

TABLE D

| Component | Composition (%)(w/w) |
|---|---|
| Glycerin | About 10 to about 50 |
| Ethyl alcohol | About 10 to about 30 |
| Polysorbate-80 | About 1 to about 5 |
| Water | About 25 to about 45 |
| Benzoic acid | About 0.05 to about 2.0 |
| BHT | About 0.01 to about 0.10 |
| HEC | About 0.1 to about 0.5 |
| Disodium EDTA | About 0.01 to about 0.1 |

In another embodiment, the formulation has a pH between about 2.5 and about 4.5 and comprises between about 1 to about 6% (w/w) terbinafine hydrochloride and one or more additional components listed in Table D in the designated amounts.

In an additional embodiment, the formulation comprises about 1 to about 10% (w/w) terbinafine hydrochloride, from about 10 to about 30% (w/w) ethanol (95% ethanol), and about 1 to about 40% polyethylene glycol 400.

In another embodiment, the formulation comprises about 1 to about 10% (w/w) terbinafine hydrochloride, from about 10 to about 30% (w/w) ethanol (95% ethanol), about 1 to about 50% glycerin.

In yet another embodiment, the formulation comprises about 1 to about 10% (w/w) terbinafine hydrochloride, from about 10 to about 30% (w/w) ethanol (95% ethanol), about 1 to about 40% polyethylene glycol 400 and about 1 to about 50% glycerin.

In an additional embodiment, the formulation comprises from about 1 to about 6% (w/w) terbinafine hydrochloride, from about 10 to about 30% (w/w) ethanol (95% ethanol) and from about 10 to about 50% (w/w) glycerin.

In yet another embodiment, the formulation comprises from about 1 to about 6% (w/w) terbinafine hydrochloride, from about 10 to about 30% (w/w) ethanol (95% ethanol), from about 10 to about 50% (w/w) glycerin and from about 0.05 to about 0.5% (w/w) benzoic acid.

In an additional embodiment, the formulation comprises about 4% (w/w) terbinafine hydrochloride, about 21% (w/w) ethanol (95% ethanol), about 40% (w/w) glycerin and about 0.2% (w/w) benzoic acid.

In yet another embodiment, the formulation comprises about 4% (w/w) terbinafine hydrochloride, about 21% (w/w) ethanol (95% ethanol), about 5% (w/w) polysorbate-80, and about 40% polyethylene glycol 400.

In a further embodiment, the formulation comprises about 4% (w/w) terbinafine hydrochloride, about 21% (w/w) ethanol (95% ethanol), about 5% (w/w) polysorbate-80, about 40% polyethylene glycol 400, 0.01% (w/w) BHT and about 0.01% EDTA.

In another embodiment, the formulation comprises about 4% (w/w) terbinafine hydrochloride, about 21% (w/w) ethanol (95% ethanol), about 5% (w/w) polysorbate-80, about 10% (w/w) glycerin, about 0.2% (w/w) benzoic acid and about 30% (w/w) polyethylene glycol 400.

In a further embodiment, the formulation comprises about 4% (w/w) terbinafine hydrochloride, about 21% (w/w) ethanol (of 95% ethanol), about 5% (w/w) polysorbate-80, about 10% (w/w) glycerin, about 0.2% (w/w) benzoic acid, about 40% (w/w) polyethylene glycol 400, 0.01% (w/w) BHT and about 0.01% EDTA.

In yet another embodiment, the formulation comprises about 4% (w/w) terbinafine hydrochloride, about 21% (w/w) ethanol (of 95% ethanol), about 5% (w/w) polysorbate-80, about 40% (w/w) glycerin, about 0.3% HEC, 0.2% (w/w) benzoic acid, about 0.01% (w/w) BHT and about 0.01% (w/w) disodium EDTA.

In an additional embodiment, the formulation comprises about 4% (w/w) terbinafine hydrochloride, about 21% (w/w) ethanol (of 95% ethanol), about 5% (w/w) polysorbate-80, about 2% (w/w) polyvinylpyrrolidone, 0.01% (w/w) BHT and about 0.01% EDTA.

In a further embodiment, the formulation comprises about 4% (w/w) terbinafine hydrochloride, about 21% (w/w) ethanol (95% ethanol), about 5% (w/w) polysorbate-80, about 30% (w/w) polyethylene glycol (PEG) 400, about 10% (w/w) glycerin and about 0.3% (w/w) HEC.

In yet another embodiment, the formulation comprises about 4% (w/w) terbinafine hydrochloride, about 21% (w/w) ethanol (95% ethanol), about 5% (w/w) polysorbate-80, about 30% (w/w) polyethylene glycol (PEG) 400, about 10% (w/w) glycerin, about 0.3% (w/w) HEC, 0.01% (w/w) BHT and 0.01% (w/w) disodium EDTA.

In an additional embodiment, the formulation comprises about 4% (w/w) terbinafine hydrochloride, about 21% (w/w) ethanol (95% ethanol), about 5% (w/w) polysorbate-80, about 30% (w/w) polyethylene glycol (PEG) 400, about 10% (w/w) glycerin, about 2% (w/w) polyvinylpyrrolidone and about 0.3% (w/w) HEC.

In an additional embodiment, the formulation comprises about 4% (w/w) terbinafine hydrochloride, about 21% (w/w) ethanol (95% ethanol), about 5% (w/w) polysorbate-80, about 30% (w/w) polyethylene glycol (PEG) 400, about 10% (w/w) glycerin, about 2% (w/w) polyvinylpyrrolidone, about 0.3% (w/w) HEC, about 0.01% (w/w) BHT and about (w/w) 0.01% disodium EDTA.

In yet another embodiment, the formulation comprises from about 5 to about 10% (w/w) terbinafine hydrochloride, 21% (w/w) ethanol (95% ethanol), 5% (w/w) polysorbate-80, 30% (w/w) polyethylene glycol and 0.3% (w/w) HEC.

In a further embodiment, the formulation comprises from about 5 to about 10% (w/w) terbinafine hydrochloride, 21% (w/w) ethanol (95% ethanol), 5% (w/w) polysorbate-80, about 30% (w/w) polyethylene glycol, about 0.3% (w/w) HEC (w/w), about 0.01% (w/w) BHT and about 0.01% (w/w) EDTA.

In an additional embodiment, the invention is directed to a method of administering an anti-fungal drug to a patient in need thereof comprising iontophoretically administering to a body surface of the patient a suitable formulation of the anti-fungal drug. In another embodiment, the invention is directed to a method of administering terbinafine to a patient in need thereof comprising iontophoretically administering to a body surface of the patient a suitable formulation of terbinafine. In one embodiment, the terbinafine is terbinafine hydrochloride. A formulation of the anti-fungal drug, such as terbinafine, may be administered to any affected body surface. Such body surfaces include, for example, the skin, the scalp, the groin, the feet, or the nails (fingernails and/or toenails). In one embodiment, the formulation is administered to the nail or to the nail and surrounding soft tissue and/or the nail and skin interface. In another embodiment, the method comprises pre-treating the nail such that delivery to the nail is increased. In another embodiment, the method comprises treating the nail with aqueous hydration, solvent or debridement before administering the anti-fungal drug to the nail. In one embodiment, the nail is pretreated with saline. In a further embodiment, the nail is pretreated with a formulation comprising one or more of the following excipients: a solvent including, but not limited to, dioxolane, dimethyl actamide, isopropyl alcohol (IPA) and ethyl acetate and/or a carrier agent including, but not limited to, an alcohol such as IPA. In one embodiment, the pretreatment formulation comprises a solvent in an amount up to about 25% (w/w). In another embodiment, the pretreatment formulation comprises a carrier agent in an amount up to about 20% (w/w).

In one embodiment, a current density sufficient for permeation of the formulation into a body surface is applied. In another embodiment, a current density sufficient for permeation into a nail plate is applied. In one embodiment, a current density from about 10 μA/cm² to about 1 mA/cm² is applied. In another embodiment, a current density from about 50 μA/cm² to about 1 mA/cm² is applied. In yet another embodiment, a current density from about 100 μA/cm² to about 500 μA/cm² is applied. In a further embodiment, a current density from about 200 μA/cm² to about 500 μA/cm² is applied. In yet another embodiment, a current density of at least about 200 μA/cm², about 400 μA/cm², about 600 μA/cm², about 800 μA/cm² or about 1 mA/cm² is applied. In a further embodiment, a current of no more than about 1 mA/cm² is applied. In another embodiment, a current of no more than about 750 μA/cm² is applied. In yet another embodiment, a current density of no more than about 500 μA/cm² is applied.

In one embodiment, a current dose (current×time) sufficient for permeation of the formulation into a body surface is administered. In an additional embodiment, a current dose of at least about 0.5 mA*min is applied. In yet another embodiment, a current dose of at least about 15 mA*min is applied. In a further embodiment, a current dose of at least about 25 mA*min is applied. In another embodiment, a current dose from about 0.5 mA*min to about 25 mA-min is applied. In a further embodiment, a current dose from about 1 mA-min to about 20 mA*min is applied. In yet another embodiment, a current dose from about 2 mA*min to about 15 mA-min is applied.

The iontophoresis can be applied for a sufficient time to achieve an effective amount of permeation. For example, a sufficient time for application is a time from about 1 minute to about 60 minutes. In one embodiment, iontophoresis is applied for a time from about 5 minutes to about 30 minutes. In yet another embodiment, iontophoresis is applied for a time from about 5 minutes to about 15 minutes. In a further embodiment, iontophoresis is applied for a time from about 10 minutes to about 30 minutes.

In another embodiment, terbinafine is iontophoretically administered to the body surface at least twice. In a further embodiment, the terbinafine can be iontophoretically administered to the body surface at least three times. In a further embodiment, the terbinafine is iontophoretically administered to the body surface at least one time per week. In another embodiment, the terbinafine is iontophoretically administered at an interval from once a week to once every four weeks. In yet another embodiment, the terbinafine is iontophoretically administered at an interval from once a week to once a year. In a further embodiment, the terbinafine is iontophoretically administered at an interval from once a week to once every six months. In an additional embodiment, the terbinafine is administered at an interval from once a week to once every sixteen weeks. In a further embodiment, the terbinafine is administered an interval from once a week to once every twelve weeks. In another embodiment, the terbinafine is administered to the body surface at an interval from once every two weeks to once every eight weeks. In yet another embodiment, the terbinafine is administered to a body surface at an interval from once every week to once every four weeks. In an additional embodiment, the terbinafine is administered to a body surface at an interval from once every four weeks to once every eight weeks. In a further embodiment, the terbinafine is administered to the body surface at an interval from once every three weeks to once every four weeks.

In one embodiment, a formulation of anti-fungal drug is administered using an iontophoretic delivery device. In another embodiment, the formulation is adsorbed into a foam material and applied to the body surface. In yet another embodiment, the formulation is preloaded into the applicator and distributed as a single use, single dose applicator for administration using an iontophoretic delivery device. Examples of iontophoretic delivery devices useful with the compositions and methods of the invention include, but are not limited to, handheld devices and devices which comprise a separate compartment as a power supply. Exemplary devices include, but are not limited to, those described in U.S. Pat. Nos. 6,148,231, 6,385,487, 6,477,410, 6,553,253, and U.S. Patent Publication Numbers 2004/0111051, 2003/0199808, 2004/0039328, 2002/0161324, and U.S. Application Ser. No. 60/743,528, all incorporated herein by reference. An example of an applicator which can be used with a formulation of the invention comprises an active electrode adhered to an open cell polymer foam or hydrogel. Another applicator which has been developed for use with a device for iontophoretic delivery of an agent to a treatment site comprises an applicator head having opposite faces and including an active electrode and a porous pad (such as a woven or non-woven polymer, for example, a polypropylene pad); a margin of the applicator head about the active electrode having a plurality of spaced projections there along; the porous pad and the applicator head being ultrasonically welded to one another about the margin of the head with the electrode underlying the porous pad; and a medicament or a medicament and an electrically conductive carrier therefor carried by the porous pad in electrical contact with the electrode. In one embodiment, the formulation is iontophoretically administered using carbon electrodes, silver-silver chloride electrodes or silver coated carbon electrodes. In a further embodiment, the applicator is a nail-only applicator wherein drug is delivered only to the nail. In another embodiment, the applicator is a nail and skin applicator wherein drug is delivered into the nail and into the surrounding skin.

In yet another embodiment, the invention is a method of treating a fungal infection in a patient suffering therefrom comprising iontophoretically administering a formulation comprising an antifungal drug. In one embodiment, the antifungal drug is terbinafine. In another embodiment, the terbinafine is terbinafine hydrochloride. In another embodiment, the fungal infection is a dermatophyte infection. In yet another embodiment, the fungal infection is selected from the group consisting of interdigital-type pedis, tinea curis, tinea capitis, tinea corporis, tinea versicolor and onychomycosis. Interdigital-type pedis is commonly known as athlete's foot and is a fungal infection of the foot. Tinea curis is commonly referred to as “jock itch” and is a fungal infection of the groin and/or gluteal cleft. Tinea corporis is commonly referred to as “ringworm” and is an infection with dermatophytes on a region of the skin other than the scalp, groin, palms, and soles. Tinea capitis is a dermatophyte infection of the scalp.

In yet another embodiment, the fungal infection is a fungal infection of the nail. In a further embodiment, the fungal infection is onychomycosis. Onychomycosis is an invasion of the nail plate and nail bed by a fungus. In one embodiment, the invention is a method of treating onychomycosis in a patient suffering therefrom comprising iontophoretically administering a formulation comprising terbinafine hydrochloride to a nail plate of the patient.

In a further embodiment, the fungal infection is tinea capitis. In one embodiment, the invention is a method for the treatment of tinea capitis in a patient suffering therefrom comprising iontophoretically administering a formulation comprising terbinafine to the patient.

The following Examples further illustrate the present invention but should not be construed as in any way limiting its scope.

### EXAMPLES

### Example 1

### Characterizing the Solubility of Terbinafine HCl in Various Solvents

In order to establish a suitable receiver fluid for use in permeation studies, the saturated solubility of terbinafine HCl at room temperature was determined in various solvents such as, water, phosphate buffered saline (PBS), PBS with 5% Tween-80 (PBS-T), citrate buffer and sodium phosphate buffer. The drug was added to each of the solvents until visual saturation was obtained and the mixtures were left at controlled temperature (19° C.) for 24 hours. The saturated drug solution was filtered and solubility of the drug was determined by UV spectrophotometer. The results are summarized in Table 1 below. As shown in Table 1, the presence of Tween-80 increased the solubility of terbinafine hydrochloride in PBS.

TABLE 1

| Solvent | pH | Solubility (mg/ml) (n = 3) |
|---|---|---|
| Distilled water | 5.8 | 6.37 |
| PBS | 7.4 | 1.32 |
| PBS-T | 6.8 | 6.02 |
| Citrate buffer | 5 | 1.81 |
| Sodium phosphate | 8.9 | −0.04 |
| (Na₂HPO₄) | | |

### Example 2

### Effect of Tween-20 and Tween-80 on Terbinafine HCl Solubility in Water

The influence of Tween-20 and Tween-80 on terbinafine HCl solubility in water and PBS was determined. The drug was added to each of the solvents until visual saturation was obtained, then left at controlled temperature (19° C.) or 24 hours. The saturated solutions were filtered and drug solubility in the filtrate was determined by UV spectrophotometer. The results of this experiment are summarized in Table 2 below.

TABLE 2

| Solvent | Solubility (mg/ml), n = 3 |
|---|---|
| Distilled water | 6.37 ± 0.31 |
| Water and 5% Tween-20 | 10.58 ± 0.66 |
| Water and 5% Tween-80 | 11.47 ± 0.64 |
| PBS | 1.32 |
| PBS and 5% Tween-20 | 5.22 ± 0.02 |
| PBS and 5% Tween-80 | 6.53 ± 0.06 |

As shown in Table 2, addition of Tween-20 and Tween-80 almost doubled the solubility of terbinafine-HCl in water. Statistically, there was no difference between drug solubility in water and Tween-20 and water and Tween-80 (p>0.05, Mann Whitney U test). Addition of Tween-20 and Tween-80 also enhanced drug solubility in PBS. In the case of PBS, addition of Tween-80 resulted in a significantly higher (p<0.05, Mann Whitney U test) increase in terbinafine HCl solubility in PBS than addition of Tween-20.

### Example 3

### Effect of Electric Current on Permeation of Terbinafine HCl Through the Hoof

The permeation experiments were conducted to optimize the parameters for iontophoretically-assisted terbinafine HCl into the hoof membrane. In these experiments bovine hoof membranes (˜0.250 mm thickness) were used as a model for human nail plates.

A. Preparation of Hooves for Sectioning into Membranes

The bovine feet (fetlock and limb below fetlock) were purchased from Chitty Wholesales, Guilford, Surrey, U.K. The feet were held in position with a vice and the hoof was cut with panel saw. The first cut was made across the top of the hoof wall, just below the coronet.

Two digits or hooves were obtained from each foot and were set aside and fetlocks discarded. The hoof wall was cut away from the body of the hoof to obtain a large section of the hoof wall with as little of the underlying tissue attached as possible. The sole of the hoof was also cut off. The interdigital surface was not used as the hoof wall in that area was too soft and thin. The hoof wall pieces were kept in a bath of cold water and the remaining hoof (bone and tissue) was discarded. The pieces of hoof wall were then inspected for integrity, those that were split and too thin or had tissue attached were discarded. The pieces that met the required standard were cut into sections, approximately 22 mm×32 mm, to fit a microtome cassette. The hoof pieces were then glued into the cassettes using Araldite Rapid® glue. A heavy weight was placed on the glued hoof sections which were then left to set overnight. The sections were cut into membranes by microtome.

### B. Permeation Experiments

The prehydrated (in water for 60 min) hoof membrane was sandwiched between the donor and receptor compartments of Franz diffusion cell (FDC). The receptor compartment was filled with receiver fluid (5 ml of PBS-5% Tween 80 (T80), water or water-T80 as indicated) and the set up was visually examined for any leaks by inverting the FDC. Two ml of drug solutions (5-10 mg/ml) or suspensions (5-10 mg/ml) were placed in the donor compartment of the Franz diffusion cell. Electrodes were placed in the FDC-anode in the donor and cathode in the receptor compartment and an electric current of 200 μA or 1 mA or another amount was applied to the 1 cm² hoof area using a current control device for 1 h (anodal iontophoresis). Aliquots (50 μl) of the receptor phase were taken every 15 minutes for the first two hours and every hour subsequently for 8 h. A last aliquot was taken at 24 h. Some experiments were conducted for longer in which case aliquots were taken daily. The aliquots removed were replaced with an equal amount of buffer, and were assayed by HPLC (Symmetry C18 column, UV 224 nm, mobile phase 60/40 1.3% triethylamine/1.0% orthophosphoric acid and acetonitrile, 1 mL/min flow rate) to measure terbinafine HCl permeation through the membrane. Control passive experiments were conducted in the same way except for the application of electric current. Data were expressed as means ±standard deviations. All experiments were performed in triplicate.

### C. Passive Versus Iontophoretic Permeation

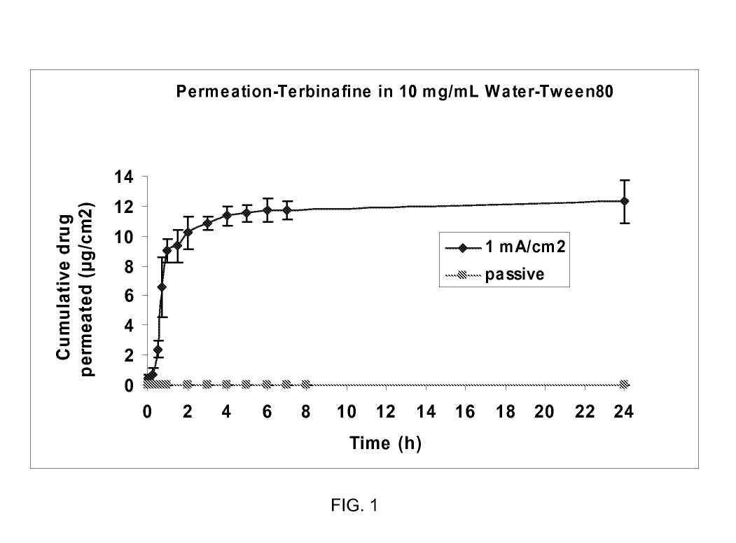

Drug permeation was measured as described above for up to 24 hours (n=3). For iontophoretic permeation, current density of 1 mA cm² was applied for 1 hr using carbon electrodes. The results of passive and iontophoretic drug permeation are shown in FIG. 1 using water-T80 and 10 mg/ml drug solution. The average of the passive replicates showed that a drug concentration of 0.02 μg/ml was achieved in the receptor phase at 24 h, suggesting low permeability. The thicknesses of the hoof membranes for three replicates were 0.292 mm, 0.243 mm and 0.282 mm. The iontophoretic drug permeation increased quite rapidly while current was applied, and then leveled off at about 8 hrs. Application of current results in repulsion between the terbinafine cations and the anode which drives the drug ions into and through the hoof membrane.

### D. Effect of Type of Electrode on the Permeation of Terbinafine HCl

The permeation experiments were conducted as described above using 1 mA/cm² for 1 hr and the following different types electrodes: stainless steel (3 mm in diameter), carbon (3 mm in diameter as graphite and the purity is 99.99%), silver-silver chloride (2 mm in diameter×4 mm pellet), gold coated copper (2 mm diameter) and platinum (25 mm×5 mm foil, purity 99.99%). Carbon and gold plated copper electrode experiments were performed using PBS-T80 and 5 mg/ml drug suspension. Silver-silver chloride, platinum, and stainless steel experiments were performed using PBS-T and 10 mg/ml drug suspension.

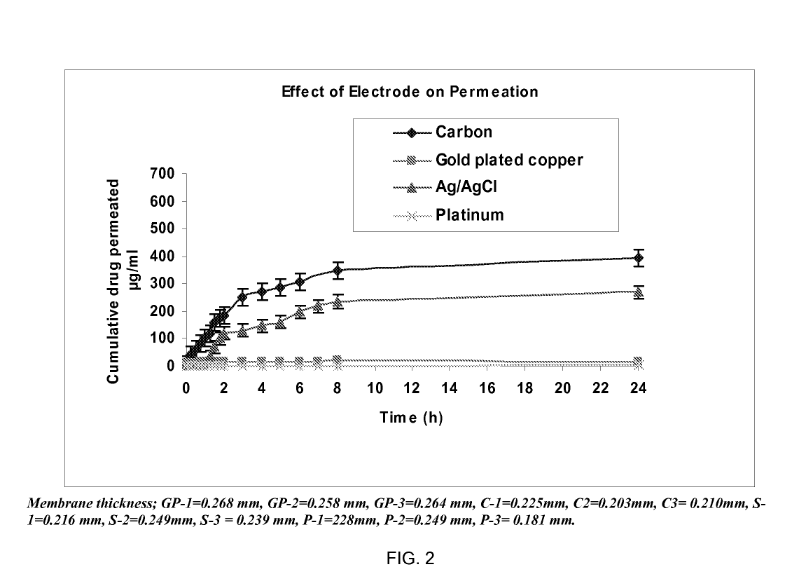

Use of stainless steel electrodes caused changes in the color of the donor solutions and was therefore not used in subsequent experiments. The permeation profiles obtained using the other electrodes are shown in FIG. 2. Higher drug concentration in the receptor phase was obtained with carbon electrodes (C1-3) and with silver-silver chloride electrodes (S1-3) than with the other electrodes tested. Use of gold plated copper electrodes (GP1-3) resulted in changes in the color of the hoof membrane, which turned to pale green color. SEM micrograph revealed that copper particles had been deposited on the membrane. Carbon electrodes showed the highest permeation profiles.

### Example 4

### Effect of Electric Current on Permeation of Terbinafine HCl Through the Nail

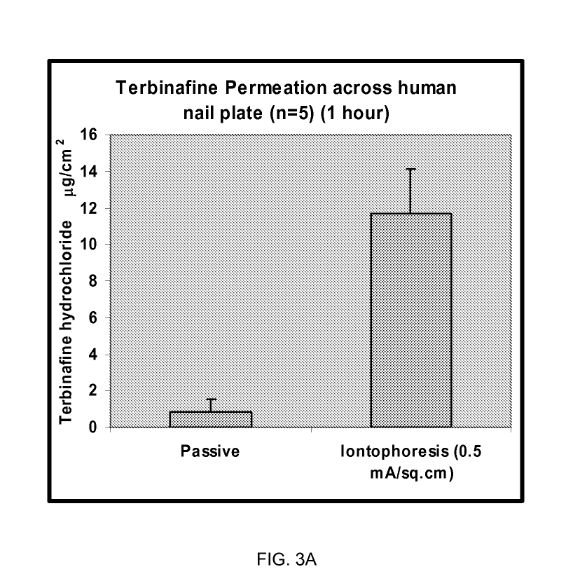

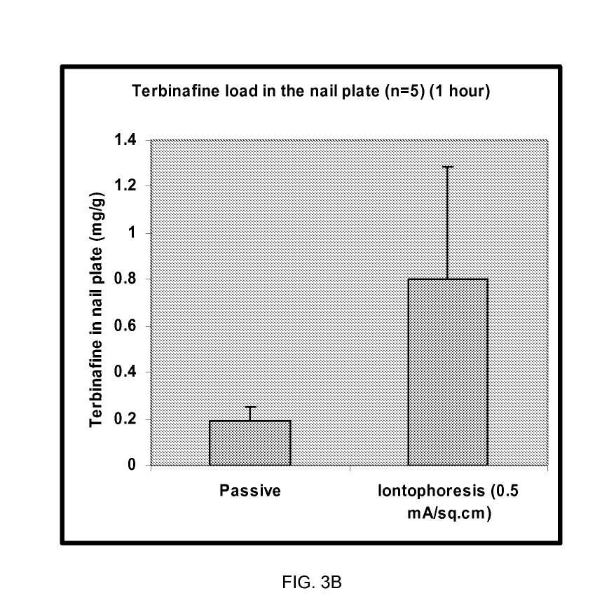

Permeation experiments were conducted using human nails to further validate iontophoretic delivery. In these experiments intact full thickness whole cadaver finger nail plates (˜0.5-0.7 mm thickness) were trimmed to appropriate disks and used. Prehydrated (in saline for 60 min) nail disks were sandwiched between the donor and receptor compartments of Franz diffusion cell (FDC) using a nail adaptor to conform to the curvature of the nail. Terbinafine hydrochloride (10 mg/mL) in water and Tween80 was used in the donor compartment, and acidified water used in the receptor. Current density of 0.5 mA/cm² was applied for 1 hr after which the receptor levels were measured for permeation of terbinafine hydrochloride through the nail using similar methods as described in Example 3B. Additionally, nail disks were analyzed (after 1 hr of iontophoresis or passive delivery) for drug in the nail using similar extraction methods as described as follows. After the permeation studies, nail membranes were removed from the Franz diffusion cells, rinsed in water and 95% ethanol several times, after which the upper surface (that had been in contact with the donor) was cleaned using cotton swab damped with 95% ethanol. The membranes were weighed and cut into small pieces for easy digestion and placed in screw cap Pyrex vials. 1.5 ml of 1N sodium hydroxide was added and the vials were incubated for 48 hours at 37° C. to allow the hoof membranes to dissolve. The vials were cooled to room temperature and 200 ul of hydrochloric acid (5N) was added to neutralize the mixture. Three ml of hexane was added to the vial to extract the drug and the vials were rotated on a roller mixer overnight. Vigorous mixing and vortexing was not selected for mixing as they led to the formation of an emulsion. The mixtures were transferred into centrifuge tubes and centrifuged at 10,000 RPM for 10 min. The upper organic layer was collected and were assayed by HPLC. Results shown in FIGS. 3A and 3B indicate that iontophoresis increases permeation through the nail and loading in the nail compared to passive delivery.

### Example 5

### Effect of Current Duration on Terbinafine-HCl Permeation Through the Nail

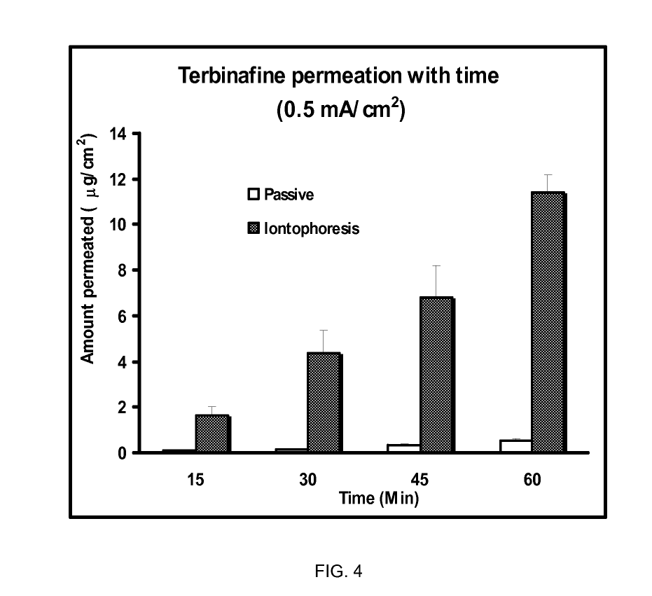

Permeation experiments were conducted using human nails as described in Example 4 but the effect of current duration was investigated. Current density of 0.5 mA/cm² was applied for either 15, 30, 45, or 60 minutes and the receptor levels analyzed for amount of terbinafine hydrochloride that permeated through the nail (μg/cm²) immediately afterwards and compared to passively delivered nails. Results indicate that there is a current duration effect on permeation of terbinafine hydrochloride through the nail (FIG. 4). Furthermore, iontophoretic permeation of terbinafine hydrochloride was greater than passively delivered drug for all current durations tested.

### Example 6

### Characterizing the Solubility of Various Anti-Fungal Compounds

The visual solubility of each receiver fluid shown in Table 3 below was initially tested for ketoconazole (BUFA B.V.) and ciclopirox (PCAS Finland Oy). Additional solubility testing was then conducted for terbinafine hydrochloride (QueMaCo Ltd.), amorolfine hydrochloride (Inter-Chemical Ltd.) and itraconazole EP (Shamrock, UK) and the results are shown in Table 3.

TABLE 3

| | Approximate visual | Approximate visual |
|---|---|---|
| | solubility (mg/ml) of | solubility (mg/ml) of |
| Receiver Fluid in PBS | ketoconazole | ciclopirox |
| 5% Ethanol | <0.91 | <0.622 |
| 10% Ethanol | <0.438 | <0.528 |
| 20% Ethanol | <0.398 | <0.225 |
| 1% Propylene glycol | <0.554 | <0.388 |
| 5% Propylene glycol | <0.472 | <0.265 |
| 0.5% Tween-80 | <0.412 | <0.446 |
| 1% Tween-80 | <0.458 | <0.452 |
| 2% Tween-80 | <0.646 | >0.728 and <0.878 |
| 5% Tween-80 | >1.28 and <1.53 | >0.158 and <1.67 |
| 20 mM B hydroxylpropyl | <1.03 | <2.02 |
| cyclodextrin | | |
| 200 mM B | >2.12 and <2.37 | >1.28 and <1.67 |
| hydroxylpropyl | | |
| cyclodextrin | | |

Ketoconazole and ciclopirox showed good solubility in receiver fluid including PBS and 5% Tween 80. The solubility of terbinafine hydrochloride, fluconazole, itraconazole and amorolfine hydrochloride was determined in PBS and 5% Tween 80 and the results are summarized in Table 4.

TABLE 4

| | Approximate visual |
|---|---|
| Drug | solubility (mg/ml) |
| Terbinafine hydrochloride | >5.00 |
| Fluconazole | >1.91 and <2.17 |
| Itraconazole | <0.33 |
| Amorolfine hydrochloride | >2.05 and <2.57 |

### Example 7

### Effect of Iontophoresis on Permeation of Ciclopirox, Amorolfine Hydrochloride and Ciclopirox Olamine Across Full Thickness Nail

The permeation experiments to determine the effectiveness of iontophoresis in enhancing the permeation of ciclopirox, amorolfine hydrochloride and ciclopirox olamine across full thickness nails were performed using a ChubTur® cell set-up (MedPharm Ltd., United Kingdom) with iontophoretic delivery or a standard ChubTur® cell. A single full thickness nail sample was positioned between the two halves of the cell and clamped together. The receptor compartment was filled with receiver fluid and the integrity of the nail/setup was determined visually by inverting the Franz cell, where leaky cells were rejected. The saturated solution (1 ml) of each compound was carefully placed into the donor compartment. For anodal iontophoresis, the power supply (dual channel Labion) of the iontophoretic device was switched on and the circuit completed by placing the cathode wire into the arm of the receiver compartment and anode wire into the donor compartment whereby the current was set at 1000 μA. After 1 hr, the iontophoretic treatment was stopped. For passive treatment, an identical protocol was followed; however, the iontophoretic device was not activated. The preliminary permeation study using the modified ChubTur® cell was sampled with multiple time points, for example, t=24, 48 hrs etc., whilst a single time point at 24 h was sampled when using the standard ChubTur® cell and assayed by determining minimum inhibitor concentration (MIC).

For assaying MIC, each 96 well plate consisting of 8 rows (letters A to H) and 12 columns (numbered 1 to 12). C. albicans Culti-Loop® (ATCC 10231, Oxoid, UK) in Ringer's solution was diluted in Sabouraud Dextrose Broth (SDB) (Oxoid, UK) to a concentration of 10⁵ CFU/ml. All the wells in columns 1 to 6 and 12 were filled with 50 μl of receiver fluid (5% Tween:PBS) with exception of wells 1A, 3A and 5A where 90 μl was added. A stock solution of each compound was prepared in receiver fluid. To the 90 μl of receiver fluid in well 1A 10 μl of the compound stock solution was added. From this, 50 μl from well 1A was mixed and transferred via a pipette in to well 1B (already containing 50 μl). Then 50 μl of well 1B was transferred to well 1C and this procedure was repeated up to and in including row 1G. Following this, 50 μl was removed from well 1G and the serial dilution was carried on from well 2A down to well 2G. The last 50 μl from well 2G was discarded. Therefore the stock solution was diluted 15 times, (each successive dilution being half the concentration of the previous well). For replicates (n=3), the entire procedure was repeated starting in well 3A and 5A.

To columns 1 to 6 and 12, 50 μl of SDB inoculated with C. albicans was added. Column 12 contained 8 positive control wells. There were also a further 8 wells containing SDB and RF with no organisms (negative control). All plates were incubated at 32° C. for 24 hrs and the opacity read at t=0 and 24 hrs on the μQuant microplate spectrophotometer 96 well plate reader at 600 nm. The results of this experiment are shown below in Table 5 (the designation “N/A” indicates that the replicate was not tested). Table 5 shows the results of permeation through full thickness human nail for ciclopirox, amorolfine and ciclopirox olamine.

TABLE 5

| | Absorbance | | |
|---|---|---|---|
| | | | Ciclopirox |
| Condition | Ciclopirox | Amorolifine | olamine |
| Positive control (no C. albicans) | 0.039 | 0.039 | 0.037 |
| Negative control (no compound) | 0.126 | 0.158 | 0.160 |
| Cathodal iontophoresis replicate 1 | 0.085 | 0.54 | 0.048 |
| Cathodal iontophoresis replicate 2 | 0.079 | 0.49 | 0.048 |
| Cathodal iontophoresis replicate 3 | 0.075 | 0.50 | N/A |
| Anodal iontophoresis replicate 1 | 0.045 | 0.54 | 0.042 |
| Anodal iontophoresis replicate 2 | 0.044 | 0.44 | 0.079 |
| Anodal iontophoresis replicate 3 | 0.068 | 0.40 | N/A |
| Passive permeation replicate 1 | 0.075 | 0.48 | 0.157 |
| Passive permeation replicate 2 | 0.046 | 0.59 | 0.129 |
| Passive permeation replicate 3 | N/A | 0.54 | N/A |

As shown above, under the conditions of this experiment, with full thickness nails, ciclopirox olamine was detected in the receiver fluid (the absorbance was similar to the positive control) with both cathodal and anodal iontophoresis, but not with passive delivery. Under the conditions of this study, ciclopirox appeared to penetrate the nail regardless of whether current was applied. In this study, amorolfine did not show permeation of the nail under any conditions tested. The results of this experiment show that iontophoresis improved the permeation of ciclopirox olamine across full thickness nails.

### Example 8

### Comparing Different Formulations of Terbinafine Hydrochloride for Drug in the Hoof Membrane and Drug that Permeates the Hoof Membrane

The following different formulations of terbinafine hydrochloride were prepared (all adjusted to pH 3):
a. 25% ethanol (95% ethanol), 5% Tween-80, 40% propylene glycol, 30% water and 8% terbinafine HCl (TF-1)
b. 25% ethanol, 5% Tween-80, 40% glycerin, 30% water and 4% terbinafine HCl (TF-5)
c. 15% ethanol, 5% Tween-80, 40% glycerin, 40% water and 5% terbinafine HCl (TF-10)
d. 25% ethanol, 5% Tween-80, 40% PEG400, 30% water and 5% terbinafine HCl (TF-11)

Prehydrated cadaver finger nails (approximately 0.5-0.7 mm thickness) or bovine hooves (approximately 0.25 mm thickness) were used for comparing different formulations as described in Examples 8-10. The nails or hooves were sandwiched between the donor and receptor compartments of Franz diffusion cell (FDC), where the permeation area was 0.2 cm² for the nail and 1.0 cm² for the hoof. A nail adaptor was required to accommodate the curvature of the nail, but not for the hoof. The receptor compartments were filled with receiver fluid (3-5 ml water pH 3) and the drug formulations described above (0.5-2 mL) were placed in the donor compartment. Electrodes were placed in the FDC (anode in the donor and cathode in the receptor compartment) and electric current (0.4 mA/cm² for hoof and 0.5 mA/cm² for nail) was applied using a current control device (Transport Pharmaceuticals, Inc., Framingham, Mass.) for 1 hr, after which experiments were terminated. Receptor levels of drug permeated were analyzed. Aliquots removed were replaced with an equal amount of buffer, and were assayed by HPLC to measure terbinafine HCl permeation through the nail or hoof. Control passive experiments were conducted in the same way except for the application of electric current. Nails or hooves were taken out of the FDC and drug extracted as described in Example 4. The drug loading levels in the nail or hoof were expressed as mg terbinafine HCl per g of nail or hoof. Data were expressed as means ±standard deviations. All experiments were performed in triplicate.

The four formulations TF-1, TF-5, TF-10 and TF-11 were evaluated for permeation of Terbinafine HCl through the hoof membrane. LAMISIL AT® Spray and 10 mg/mL Terbinafine HCl in water and 5% Tween-80 (water-T80) were used as control formulations. All components were expressed as w/w.

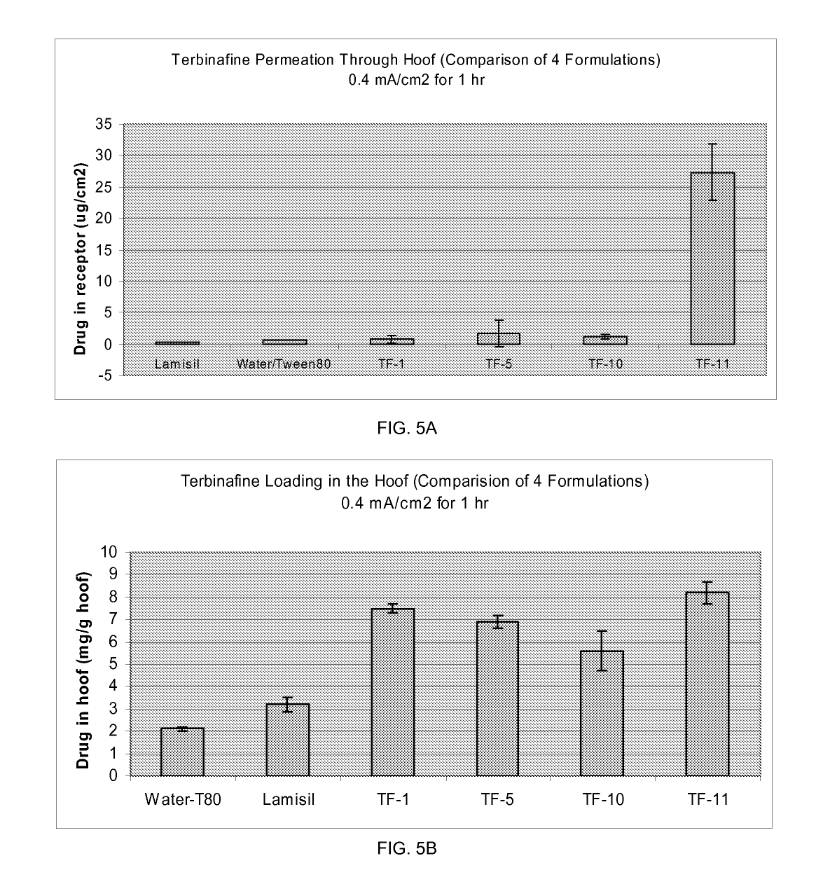

All formulations in combination with iontophoresis exhibited an increase in permeation over passive delivery (data not shown). In particular, iontophoretic delivery of glycerin and propylene glycol based formulations (TF-1, TF-5, TF-10) exhibited a moderate increase over Lamisil (FIG. 5A). As shown in FIG. 5A, the PEG400 formulation (TF-11) exhibited the highest permeation among the formulations tested.

The same TF-1, TF-5, TF-10 and TF-11 were analyzed for terbinafine HCl loading in the hoof. As shown in FIG. 5B, all four formulations exhibited high terbinafine HCl loading levels in the hoof compared to LAMISIL and water-T80. Levels were in far excess of the 0.5 μg/g observed in the nail after 4 weeks of 250 mg oral LAMISIL tablets (Faergemann et al., Acta Derm. Venereol. (1993) 73:305-309). In these formulations, the majority of the total drug delivered was concentrated in the hoof, suggesting depot formation. The amount of drug in the hoof was comparable for the four formulations of terbinafine HCl (about 7.5 mg/g for TF-1, about 7 mg/g for TF-5, about 5.5 mg/g for TF-10 and about 8 mg/g for TF-11).

### Example 9

### Hoof Permeation and Loading of Formulations Containing Permeation Enhancers

Another set of formulations were prepared containing 40% glycerin formulations with the addition of various permeation enhancers, as shown below.
a. 4.5% terbinafine HCl, 40% glycerin, 25% ethanol, 5% Tween-80, 0.3% HEC (TPI-160)
b. 4.5% terbinafine HCl, 40% glycerin, 25% ethanol, 5% Tween-80, 0.3% HEC, 0.1% cysteine (TPI-166)
c. 4.5% terbinafine HCl, 40% glycerin, 25% ethanol, 5% Tween-80, 0.3% HEC, 0.05% benzoic acid (TPI-166-2)
d. 4.5% terbinafine HCl, 40% glycerin, 25% ethanol, 5% Tween-80, 0.3% HEC, 0.5% urea (TPI-166-3)

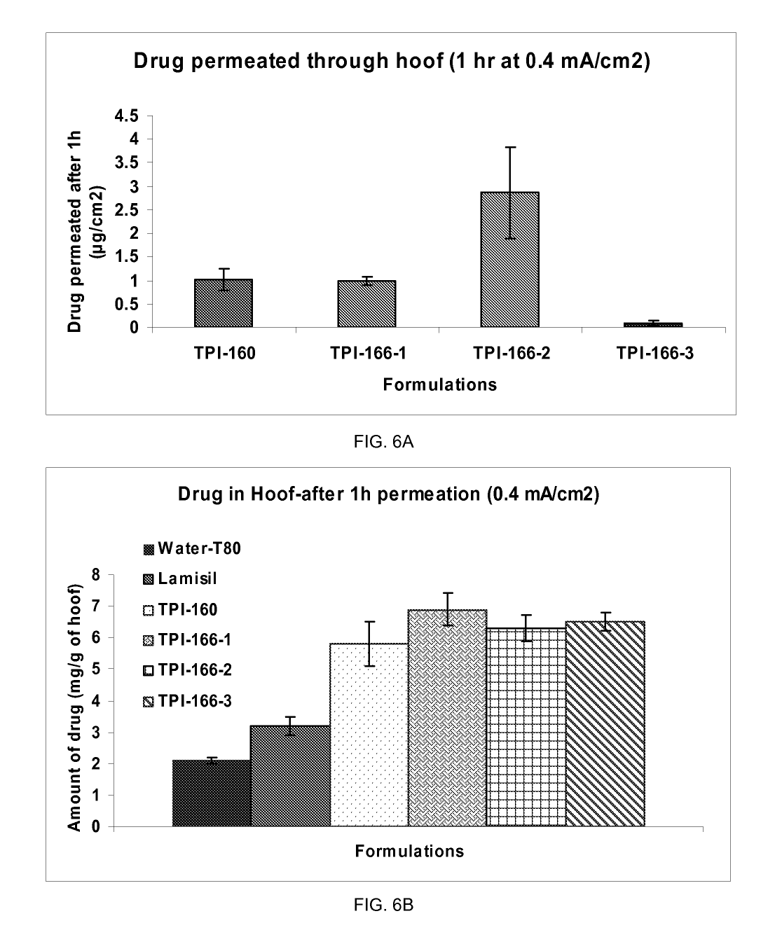

Hoof permeation was studied as described in Example 8. Compared to the base glycerin formulation (TPI-160), cysteine and urea did not seem to enhance permeation under iontophoretic delivery. However, benzoic acid (TPI-166-2) enhanced the permeation of terbinafine HCl through the hoof (FIG. 6A). Drug loading of these formulations were comparable to formulations TF-1, TF-5, TF-10 and TF-11 in Example 8 and were greater than LAMISIL and water-T80 (FIG. 6B).

### Example 10

### Nail Permeation and Loading of Terbinafine HCl Formulations

Four different formulations containing permeation enhancers, co-solvents, and antioxidants, as described below, were evaluated in the nail for permeation and loading after passive or iontophoretic delivery at 0.5 μg/cm² for 1 hr.
a. 0% BA (benzoic acid): 4% terbinafine HCl, 21% ethanol (95%), 5% Tween-80, 40% glycerin, 0.3% HEC, pH 3 (base glycerin formulation, Lot BCL336-007-5)
b. 0.2% BA: 4% terbinafine HCl, 21% ethanol (95%), 5% Tween 80, 40% glycerin, 0.3% HEC, 0.2% benzoic acid, pH 3 (BA formulation)
c. 0.2% BA+BHT+EDTA: 4% terbinafine HCl, 21% ethanol (95%), 5% Tween 80, 40% glycerin, 0.3% HEC, 0.2% benzoic acid, 0.01% BHT, 0.01% disodium EDTA, pH 3 (BA+BHT+EDTA formulation, Formulation TPI-DF-507)
d. PEG 400: 4% terbinafine HCl, 21% ethanol (95%), 5% Tween 80, 30% PEG 400, 10% glycerin, 0.3% HEC, pH 3 (PEG400 formulation)

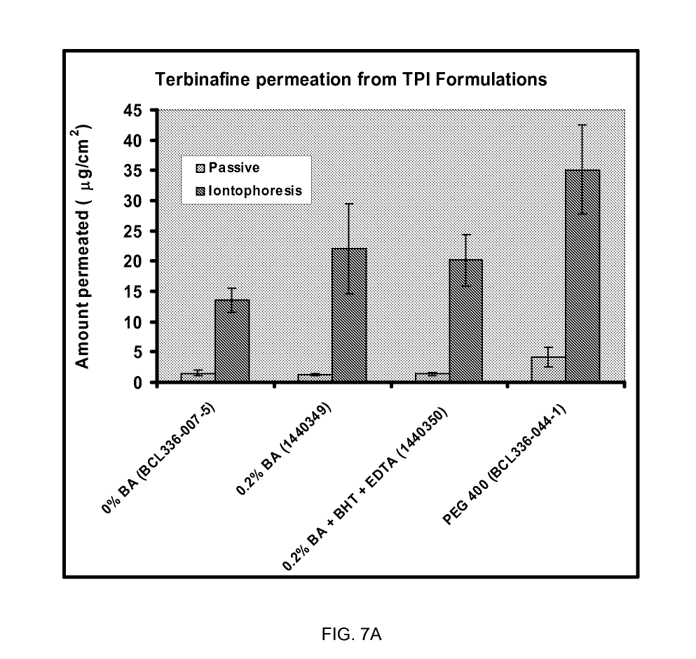

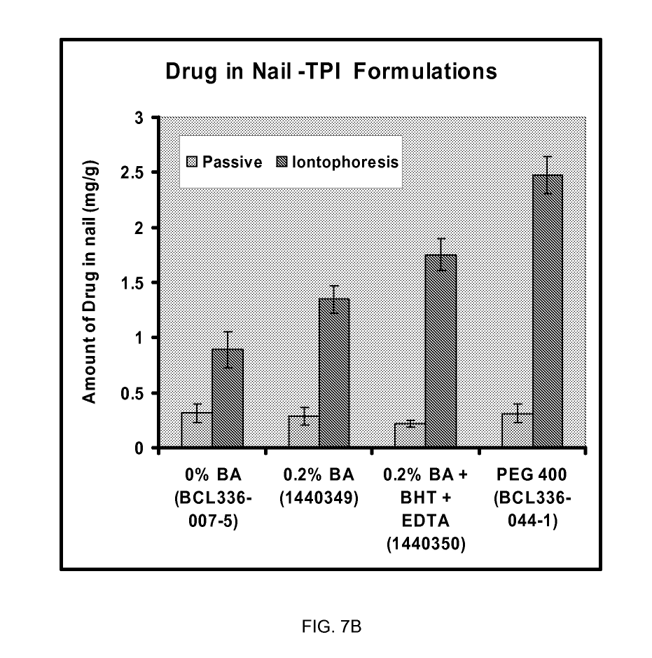

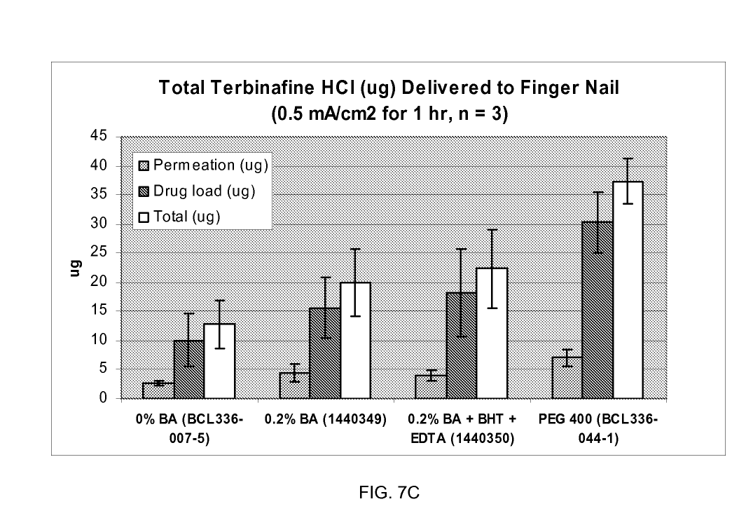

The four formulations were evaluated for permeation of terbinafine HCl through the nail and loading in the nail as described in Example 8. Formulations containing benzoic acid improved permeation of terbinafine HCl through the nail over the base glycerin formulation without benzoic acid (FIG. 7A). The addition of antioxidants such as BHT and EDTA did not affect this enhanced permeation. Formulation containing PEG 400 further improved the permeation of terbinafine HCl through the nail. In all cases, iontophoresis clearly increased permeation of terbinafine HCl through the nail when compared to passive delivery.

Drug loading in the nail ranged from 0.8-2.5 mg terbinafine HCl per gram (g) of nail for the four formulations tested (FIG. 7B). Addition of benzoic acid seemed to improve drug loading over the base glycerin formulation. Addition of PEG 400 seemed to increase drug loading further. All four formulations achieved drug loading levels in far excess of levels achieved with oral LAMISIL as described in Example 8. These high loading levels suggest a depot formation in the nail, in addition to drug permeating through the nail. When terbinafine HCl levels that had permeated through the nail were compared with what was in the nail after 1 hr of iontophoresis, the drug-in-nail levels were much greater than drug in the receptor, suggesting that much of the drug accumulates in the nail and may act as a reservoir for sustained release over longer periods of time. Drug loaded in the nail ranged between 78-94% of the total delivered drug (FIG. 7C). In all cases, iontophoresis clearly increased loading of terbinafine HCl in the nail when compared to passive delivery. These results indicate a combination of permeation (which may drive the drug down to the nail bed initially) and loading (which may serve as a reservoir for sustained release of drug over time).

### Example 11

### Stability Study of Terbinafine Hydrochloride Formulation at 55° C.

The stability of the five formulations described below was studied for 4 weeks at 55° C. Stability was determined each week using a HPLC assay procedure for the determination of terbinafine HCl, its related substances and degradation products in gel with detection by UV absorption at 220 nm. The results of the study are shown below in Table 6.

Each of the formulations contains 4% terbinafine hydrochloride, 40% glycerin, 21% ethanol (95% ethanol), 5% polysorbate-80, 0.3% HEC and the components described in the first column of the Table below.

TABLE 6

| Formulation | Weeks | Percent Area |
|---|---|---|
| 0.2% Benzoic acid | 1 | 100 |
| | 2 | 100 |
| | 3 | 99.47 |
| | 4 | 99.42 |
| 0.2% Benzoic acid | 1 | 100 |
| 0.01% BHT | 2 | 100 |
| | 3 | 100 |
| | 4 | 99.93 |
| 0.2% Benzoic acid | 1 | 100 |
| 0.01% EDTA | 2 | 100 |
| | 3 | 100 |
| | 4 | 99.96 |
| 0.2% Benzoic acid | 1 | 100 |
| 0.01% BHT | 2 | 100 |
| 0.01% EDTA | 3 | 100 |
| | 4 | 100 |
| Base glycerin | 1 | 100 |
| formulation | 2 | 100 |
| (absence of BA, | 3 | 100 |
| EDTA or BHT) | 4 | 99.89 |

### Example 12

### Effect of Current Density and Duration on Terbinafine Hydrochloride Permeation Through and Loading into the Nail

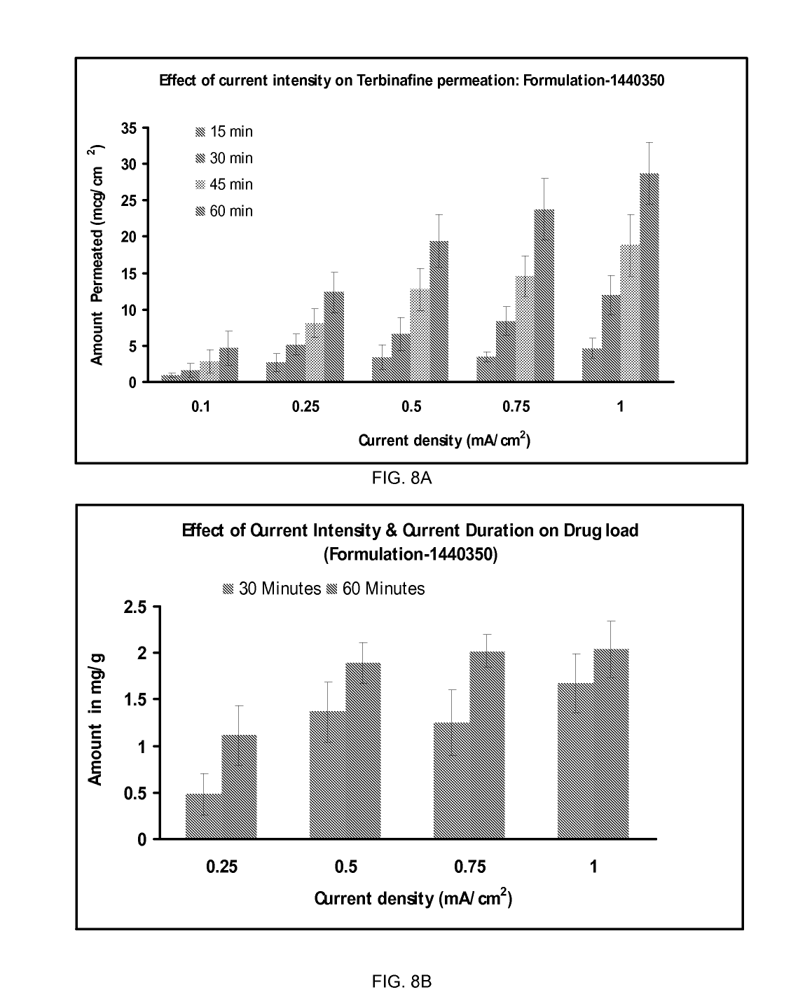

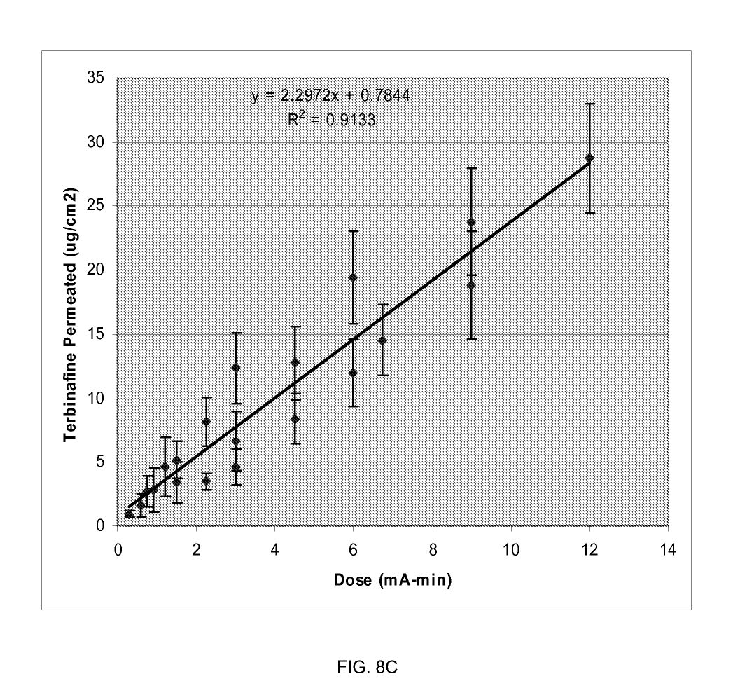

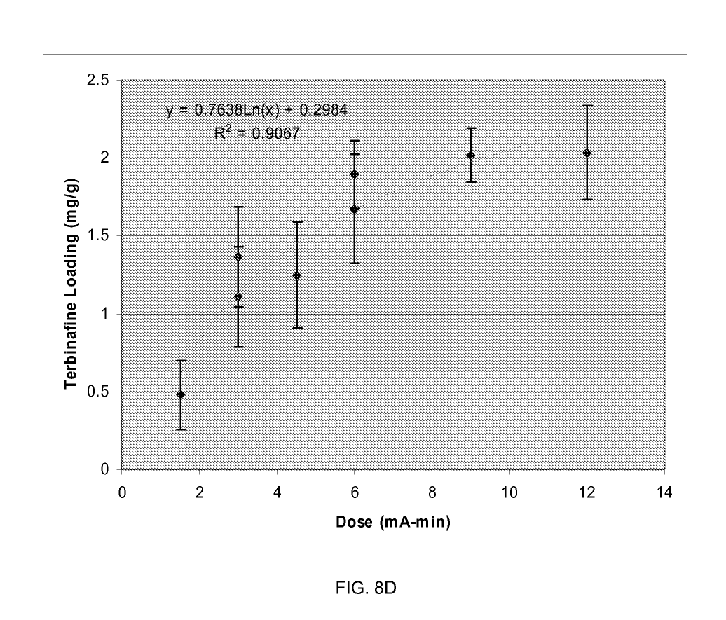

The effect of current density on the permeation of terbinafine from a formulation containing 4% terbinafine HCl, 21% ethanol (95%), 5% Tween 80, 40% glycerin, 0.3% HEC, 0.2% benzoic acid, 0.01% BHT, and 0.01% disodium EDTA (Formulation TPI-DF-507, Lot 1440350) was evaluated at different current densities (0.1, 0.25, 0.5, 0.75 and 1 mA/cm²) and time intervals (15, 30, 45 and 60 minutes). The effect of current density on the drug nail loading from the same formulation was carried out at current densities of 0.25, 0.5, 0.75 and 1 mA/cm² at two different time intervals (30 and 60 minutes). The results indicate that there is a current duration and current density effect on permeation of terbinafine hydrochloride through the nail (FIG. 8A). In addition, drug loading levels also increased with increasing current duration at all current densities evaluated, although the differences were generally small, especially at the higher current densities (FIG. 8B). Drug loading did not increase further beyond 0.5 mA/cm² suggesting that a maximum drug loading had been achieved (FIG. 8B). These results suggest that terbinafine HCl delivery is linearly dependent on total Coulombic dose of current delivered (current multiplied by duration) and that a particular set of iontophoretic conditions can be used to target desired drug delivery levels. For the Franz diffusion cell system with a 0.2 cm² active diffusion area, drug permeation and loading with respect to Coulombic dose (mA*min) is shown in FIGS. 8C and 8D, respectively. There was a linear drug permeation with increasing Coulombic dose but approaching saturation drug loading at about 6 mA*min.

### Example 13

### Evaluation of Terbinafine Hydrochloride Delivery from Two Types of Applicators Using a Nail on Agarose Model

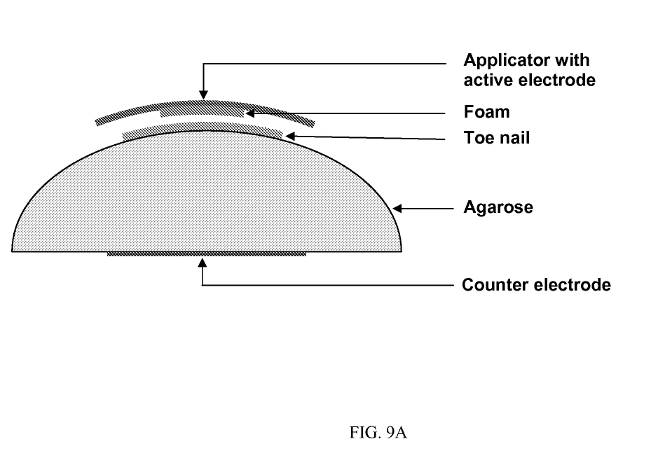

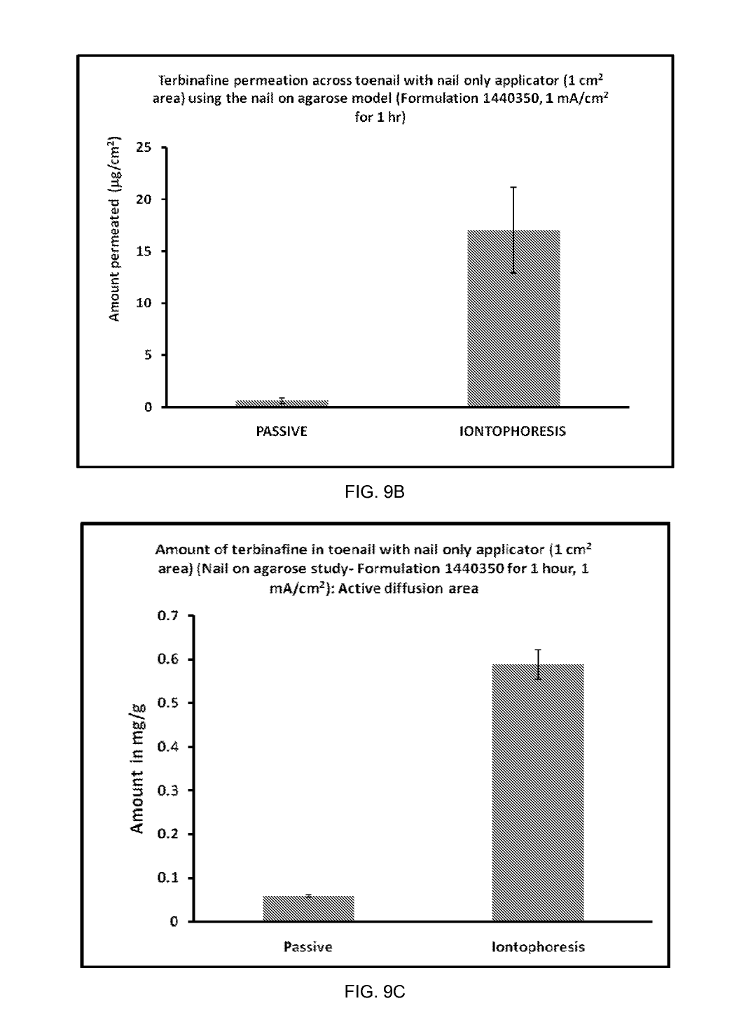

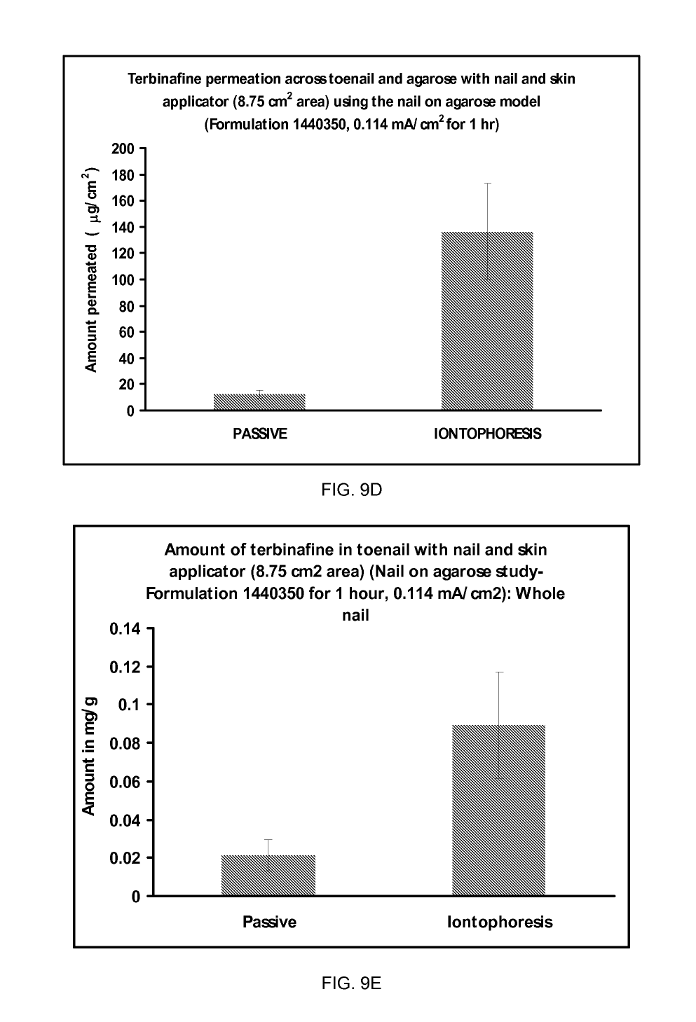

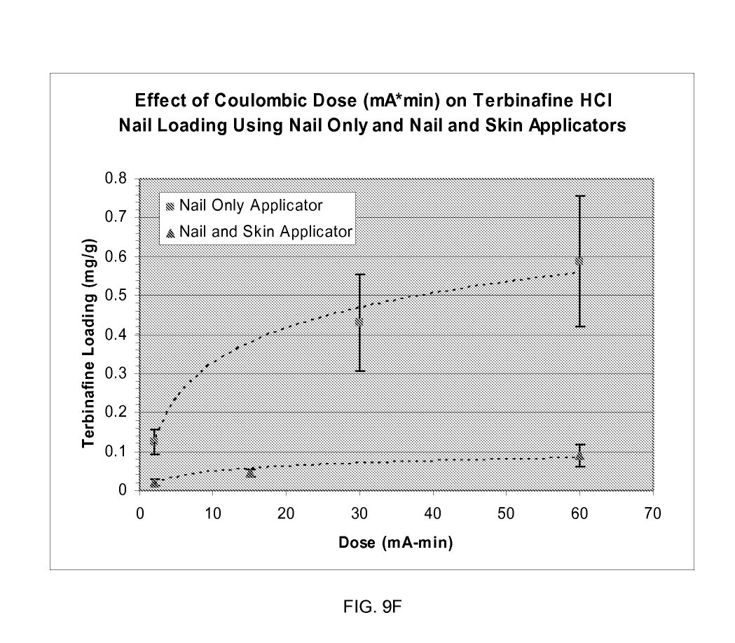

Terbinafine HCl delivery from two types of applicators was assessed using an intact nail on agarose model as shown in FIG. 9A. FIG. 9A shows a drawing of the nail-only applicator where the drug was delivered only to the nail. In the nail and skin applicator, the drug was delivered to both the nail and the skin (not shown). In the nail and skin applicator, the polyurethane foam and the active electrode (Ag—C) extended past the toe nail and onto agarose to mimic skin delivery. The agarose was covered with fluorinated ethylene propylene (FEP) membrane with uniform 0.5 mm holes distributed 5 mm apart to mimic the semi-restrictive property of the skin. The formulation containing 4% terbinafine HCl, 21% ethanol (95%), 5% Tween 80, 40% glycerin, 0.3% HEC, 0.2% benzoic acid, 0.01% BHT, and 0.01% disodium EDTA (Formulation TPI-DF-507, 80 μL and 875 μL for the nail only and nail and skin applicators, respectively) was loaded on to the polyurethane foam (1 cm² and 8.75 cm² on the nail only and nail and skin applicators, respectively) and then placed on the toe nail. The applicator extensions were adhered down to the base of the glass plate where the agarose was placed. The saline presoaked (1 hr) toe nail was placed on the curved agarose gel (1% w/v) with a counter electrode on the bottom of the agarose (standard EKG AgCl electrode with hydrogel, Vermed). Three replicates were run for 1 hr at a total current of 1 mA (1 mA/cm² and 0.114 mA/cm² for the nail only and nail and skin applicators, respectively, and both at 60 mA*min Coulombic dose). Passive delivery was also run for 1 hr without current. At the end of 1 hr of delivery, the active diffusion area of the nail (1 cm² area and approximately 4 cm² whole nail area using the nail only and nail and skin applicators, respectively) was extracted for drug loading using the standard extraction procedure. The amount of drug permeated through the nail and into the agarose was determined by extracting terbinafine using 0.1M HCl and analyzing the samples by HPLC.

Using the nail-only applicator, permeation of terbinafine HCl through the toe nail and into the agarose with iontophoresis was comparable to previous results and far greater than passive delivery levels, further validating that iontophoretic delivery of terbinafine HCl is able to drive the drug through the nail (FIG. 9B). Terbinafine HCl loading in the nail was also significantly higher than levels achieved by passive delivery as well as levels achieved in the nail by oral LAMISIL (Faergemann et al.) as discussed previously (FIG. 9C). Approximately 66% of the total delivered dose was loaded into the nail, suggesting a depot formation that may act as a reservoir for prolonged efficacy.

Using the nail and skin applicator, permeation of terbinafine HCl through both the toe nail and the agarose with iontophoresis was higher than levels delivered from the nail only applicator and also greater than passive delivery levels (FIG. 9D). The high level of permeation obtained with the nail and skin applicator was due to terbinafine delivered directly through the lower impedance area around the nail. Approximately 92% of the total permeated drug was due to drug which passed through areas surrounding the nail, whereas 8% of the permeated drug had passed through the nail. Terbinafine HCl loading in the nail was also significantly higher than levels achieved by passive delivery (FIG. 9E). In this case, the drug loading levels were measured in the whole nail (as opposed to the active diffusion area measured for the nail only applicator) and hence the average loading values were lower. In this model system, although approximately 2% of the total delivered dose was loaded into the nail and the rest into the agarose, the drug loading levels in the nail were still significantly higher than levels achieved in the nail by oral LAMISIL (Faergemann et al.).

Additional terbinafine HCl delivery testing using both types of applicators at different Coulombic doses were performed on the nail on agarose model. For the nail only applicator, Coulombic doses of 2 mA*min and 30 mA*min were evaluated (0.1 mA for 20 min and 0.5 mA for 60 min, respectively). For the nail and skin applicator, Coulombic doses of 2 mA*min and 15 mA*min were evaluated (0.1 mA for 20 min and 0.5 mA for 30 min, respectively). These additional doses, along with the higher 60 mA*min dose, were plotted as terbinafine loading (mg/g) with respect to Coulombic dose (FIG. 9F). The nail only applicator and nail and skin applicator exhibited nail loading ranges of 124-588 μg/g and 20-90 μg/g within Coulombic dose ranges of 2-60 mA*min, respectively. In all Coulombic doses tested in both types of applicators, terbinafine loading was significantly higher than levels achieved in the nail by oral LAMISIL (Faergemann et al.).

### Example 14

### Evaluation of Terbinafine HCl Delivery into Hairless Rat Skin in Vitro

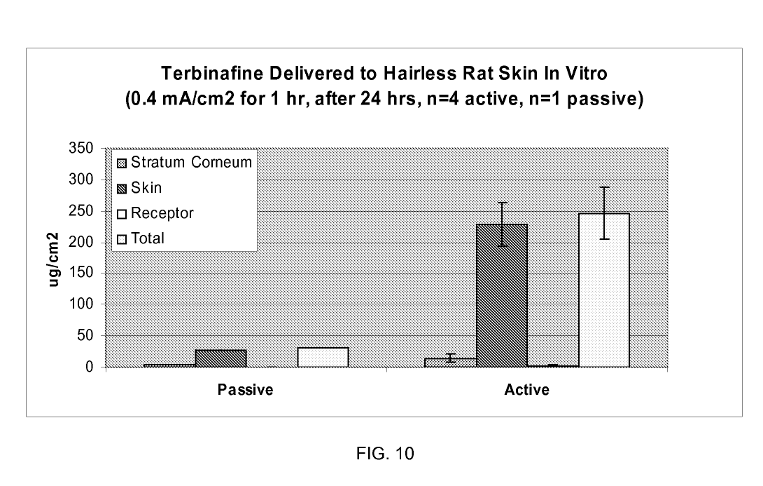

Terbinafine HCl delivery into full thickness hairless rat skin in vitro (using a 0.65 cm² diffusion area FDC) was performed to characterize the effect of iontophoresis on skin delivery of terbinafine HCl. The formulation containing 4% terbinafine HCl, 21% ethanol (95%), 5% Tween-80, 40% glycerin, 0.3% HEC, 0.2% benzoic acid, 0.01% BHT, and 0.01% disodium EDTA (Formulation TPI-DF-507, 0.5 ml) was placed in the donor in contact with the stratum corneum layer of the skin. Receptor fluid (5 mL) consisted of 10% (v/v) methanol, 30% propylene glycol, and 60% 10 mM NaCl. Silver electrode was used in the donor (and Ag/AgCl electrode in receptor) for anodal iontophoresis (0.4 mA/cm² for 1 hr) and compared with passive delivery. Receptor fluid levels were obtained throughout the 24 hr study to determine the amount of drug permeation through the skin. After 24 hrs (1 hr of current, followed by 23 hrs of passive diffusion), the skin was tape stripped (12 strips) to remove the stratum corneum, drug was extracted using 0.01 N HCl, and analyzed for terbinafine content using HPLC. The underlying skin was also extracted using 1.5 ml of 5 N NaOH and 0.1 ml of methanol heated for 1.5 hrs in a water bath maintained at 90° C. to hydrolyze the skin. The hydrolysates were neutralized by the addition of 0.45 ml H₃PO₄, followed by the addition of 2 ml of receptor. The samples were then centrifuged and the supernatant analyzed using HPLC. The total amount of drug delivered to the skin was significantly higher in the iontophoresis (active) group compared to passive delivery after 24 hrs (FIG. 10). The majority of the total delivered drug (93%) was in the skin, with the remaining drug in the stratum corneum. Almost none of the delivered drug was found in the receptor fluid, indicating that terbinafine HCl may be forming a depot and may not readily migrate into the deeper layers of the skin.

### Example 15

### In Vitro Release of Terbinafine HCl from Drug Loaded Nails

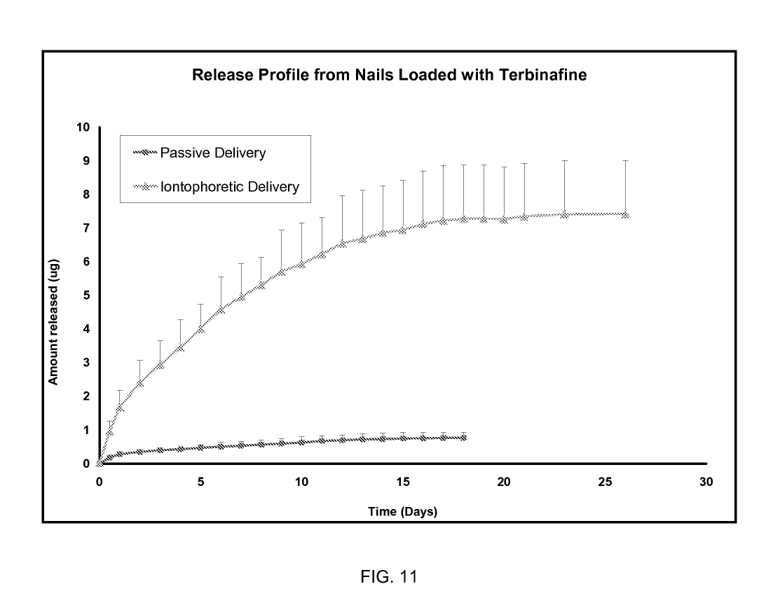

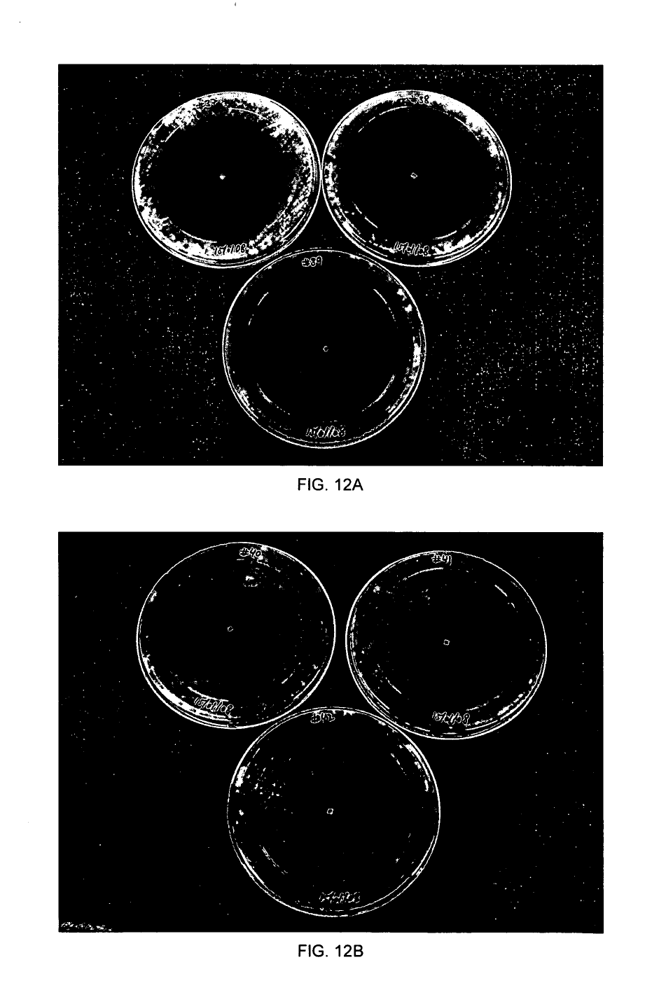

In vitro release of terbinafine HCl from nails subjected to iontophoretic and passive delivery was followed for approximately 4 weeks. Nails soaked in normal saline for one hour were mounted on a nail adapter and sandwiched between the two chambers of the FDC. The formulation containing 4% terbinafine HCl, 21% ethanol (95%), 5% Tween 80, 40% glycerin, 0.3% HEC, 0.2% benzoic acid, 0.01% BHT, and 0.01% disodium EDTA (0.5 mL) was placed in the donor compartment and pH 3 water was placed in the receiver compartment. Anodal iontophoresis was carried out at 0.5 mA/cm² for 1 hr using a custom made constant DC power supply. For comparison, passive delivery of the same formulation was performed for 1 hr. For drug release studies, nails loaded with drug were washed to remove the surface drug and were mounted on FDCs. The receiver compartment was filled with pH 3 water. The donor chamber was empty but was covered with parafilm. The receiver compartment medium was sampled daily and the amount of drug released from the drug loaded nails was measured until the cumulative release attained a plateau. Drug delivered into the nail via iontophoresis released terbinafine HCl in a sustained release fashion over approximately 3 weeks at levels far greater than passively loaded nails which reached a plateau only after a few days (FIG. 11). The amount of drug released from iontophoretically loaded nails represented approximately 47% of the initial drug loaded. Undelivered drug was confined to the upper layers of the nail and was hypothesized to be either entrapped or keratin bound. These results suggest that iontophoretically drug loaded nails serve as depots from which drug is slowly released over time. Actual release may be slower in vivo since the nail is not in contact with a fluid reservoir with enhanced drug solubility and infinite sink conditions.

### Example 16

### Antifungal Activity of Terbinafine Loaded Nails Using an Agar Diffusion Assay

The nail plates loaded with the formulation containing 4% terbinafine HCl, 21% ethanol (95%), 5% Tween-80, 40% glycerin, 0.3% HEC, 0.2% benzoic acid, 0.01% BHT, and 0.01% disodium EDTA (Formulation TPI-DF-507), by passive diffusion (15 or 60 min) or iontophoresis (Coulombic doses of 0.75, 3, 6, and 12 mA min corresponding to 0.25 mA/cm² for 15 min, 0.25 mA/cm² for 60 min, 0.5 mA/cm² for 60 min, and 1 mA/cm² for 60 min, respectively), were tested for antifungal activity. The inoculum, T. rubrum, (ATCC10218) was prepared in sterile saline from 7-10 days old cultures on PDA slants at 30° C. Terbinafine HCl loaded nail pieces (2˜3 mm squares) were placed on the Sabouraud Dextrose Agar plates (Oxoid, Napean, Ontario, Canada) inoculated with T. rubrum. The diameter (mm) of the zones of inhibition were measured using a dial caliper after 4 days of incubation at 30° C. and were compared with the standards. The zone of inhibition for the standards (known drug concentrations: 0.1, 0.2, 0.4, 0.5, 0.6, 0.8, 1.0. 2.0 and 3.0 μg of terbinafine HCl) were determined similarly by loading the drug on 6 mm paper blank disks (Becton Dickinson, Sparks, Md.). Subsequently, the terbinafine HCl standard curve generated was used to calculate the drug dosage out of the nail. Furthermore, in one set of samples (0.5 mA/cm² for 60 min and corresponding passive), a passaging study was performed in which the nail samples were transferred to fresh plates after each 4 day incubation period. Nail samples were transferred to fresh inoculated plates until no further zone of inhibition was observed. Based on the passaging study, duration of release of active terbinafine HCl was determined.

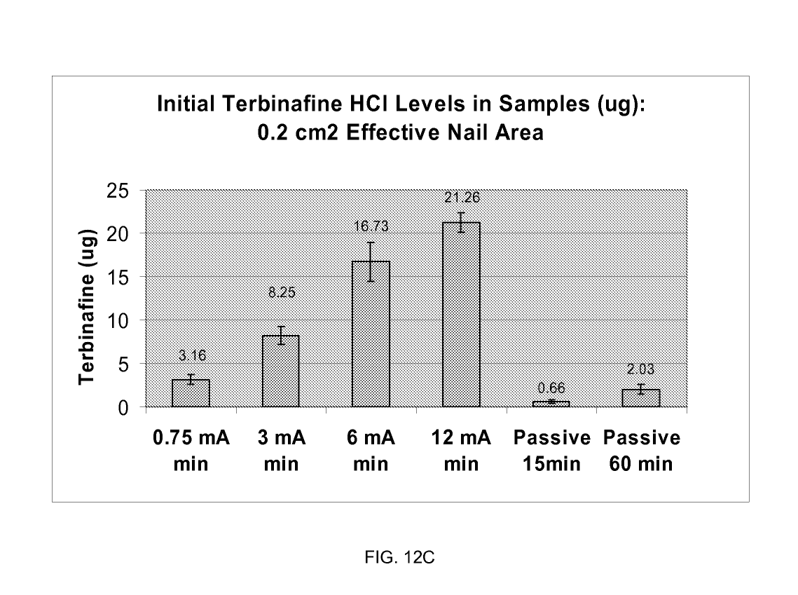

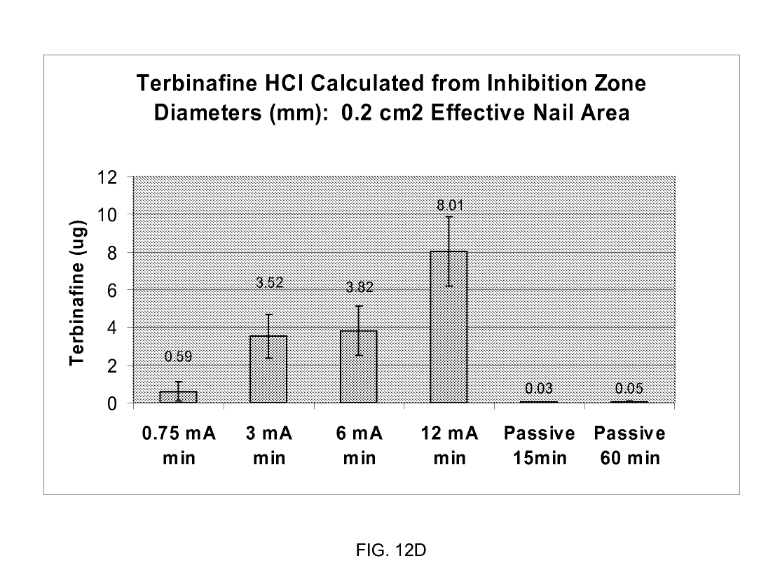

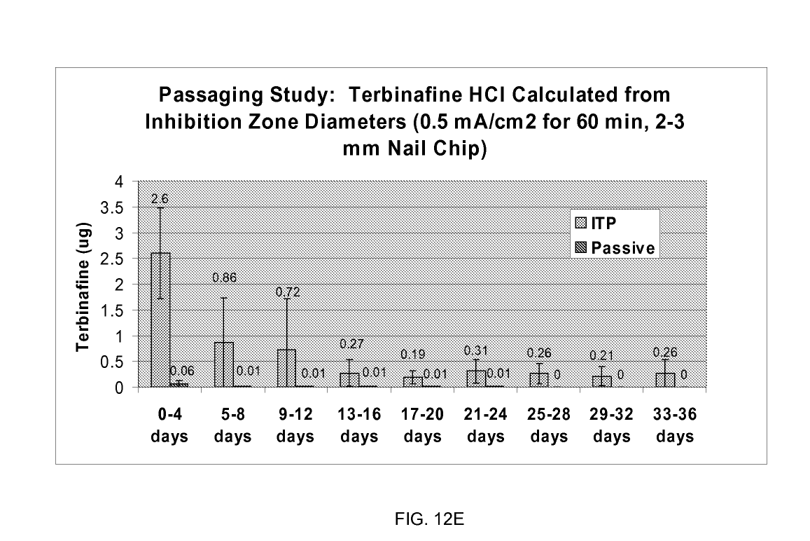

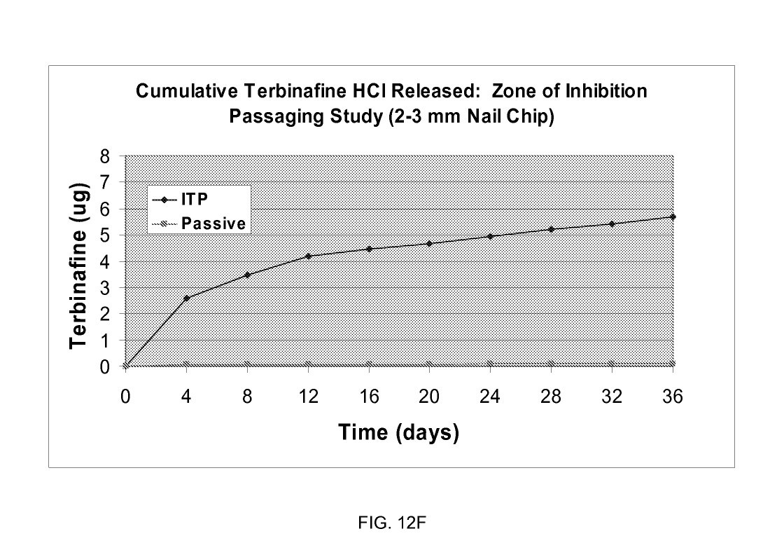

Significantly larger zones of inhibition were observed for the iontophoresis loaded nails as compared to the passive delivery controls (FIGS. 12A and 12B). The initial doses of terbinafine HCl loaded nail samples followed a dose dependent effect with respect to increasing Coulombic dose applied (from 0.75 mA*min to 12 mA*min) (FIG. 12C). Nails treated with iontophoresis exhibited a much higher drug loading overall compared to passive. After the 4 day agar diffusion study, the iontophoretically loaded nails exhibited inhibition zone diameters that increased with increasing Coulombic dose and far greater than zones formed by passive. Terbinafine HCl calculated from the inhibition zone diameters increased with increasing Coulombic dose for the iontophoretically treated groups and were far greater than passive levels (FIG. 12D). Duration of terbinafine HCl release from nail samples, as demonstrated by serial passaging of nail samples to freshly inoculated plates, persisted up to at least 36 days and is likely to continue releasing active terbinafine HCl at levels significantly higher than its MIC (0.004 μg/mL) (FIGS. 12E and 12F).

While this invention has been particularly shown and described with references to preferred embodiments thereof, it will be understood by those skilled in the art that various changes in form and details may be made therein without departing from the scope of the invention encompassed by the appended claims.

What is claimed is:

1. A formulation suitable for iontophoresis comprising terbinafine hydrochloride, one or more solvents or co-solvents and a permeation enhancer.

2. The formulation of claim 1 wherein the terbinafine hydrochloride is present in an amount from about 1 to about 10% (w/w).

3. The formulation of claim 2 wherein the one or more solvent or co-solvents are each present in an amount from about 10 to about 50% (w/w).

4. The formulation of claim 3 wherein said formulation comprises water, an alcohol and glycerin.

5. The formulation of claim 1 wherein the permeation enhancer is selected from the group consisting of benzoic acid, oleic acid, salicylic acid, cysteine hydrochloride, N-acetylcysteine and urea.

6. The formulation of claim 1 wherein the permeation enhancer is present in an amount from about 0.05 to about 50% (w/w).

7. The formulation of claim 5 wherein the permeation enhancer is benzoic acid.

8. The formulation of claim 7 wherein the benzoic acid is present in an amount from about 0.05 to about 5% (w/w).

9. The formulation of claim 8 wherein the benzoic acid is present in an amount of about 0.2% (w/w).

10. The formulation of claim 6 wherein the permeation enhancer is polyvinylpyrrolidone.

11. The formulation of claim 6, wherein the permeation enhancer is a polyethylene glycol.

12. The formulation of claim 11, wherein the polyethylene glycol is polyethylene glycol 400.

13. The formulation of claim 1 further comprising an antioxidant.

14. The formulation of claim 13 wherein the antioxidant is butylated hydroxytoluene (BHT).

15. The formulation of claim 1 further comprising a chelating agent.

16. The formulation of claim 15 wherein the chelating agent is disodium EDTA.

17. The formulation of claim 1 further comprising a viscosity modifying agent.

18. The formulation of claim 17 wherein the viscosity modifying agent is hydroxyethylcellulose (HEC).

19. The formulation of claim 1 further comprising a non-ionic surfactant.

20. The formulation of claim 19 wherein the non-ionic surfactant is selected from the group consisting of polysorbate-20, polysorbate-40 and polysorbate-80.

21. The formulation of claim 15 wherein the chelating agent is present in an amount from about 0.01 to about 0.10% (w/w).

22. The formulation of claim 13 wherein the antioxidant is present in amount from about 0.005 to about 0.10% (w/w).

23. The formulation of claim 17 wherein the viscosity modulating agent is present in an amount from about 0.10 to about 0.50% (w/w).

24. The formulation of claim 19 wherein the non-ionic surfactant is present in an amount from about 1 to about 10% (w/w).

25. The formulation of claim 1 wherein the pH of the formulation is about 2.5 to about 4.5.

26. The formulation of claim 1 comprising terbinafine hydrochloride, ethanol, glycerin and benzoic acid.

27. The formulation of claim 26 comprising about 4% (w/w) terbinafine hydrochloride, about 0.2% (w/w) benzoic acid, about 21% (w/w) ethanol and about 40% (w/w) glycerin.

28. The formulation of claim 27 further comprising a polysorbate in an amount of about 5% (w/w), wherein the polysorbate is polysorbate-80.

29. The formulation of claim 28 further comprising about 0.01% (w/w) butylated hydroxytoluene (BHT), about 0.01% (w/w) disodium EDTA, about 5% (w/w) polysorbate-80 and about 0.3% HEC.

30. A method of administering terbinafine to a patient in need thereof comprising iontophoretically administering to a body surface of said patient the formulation of claim 1.

31. The method of claim 30, wherein the terbinafine is terbinafine hydrochloride.

32. The method of claim 31, wherein a current density of at least about 10 μA/cm² is applied.

33. The method of claim 31 wherein a current dose from about 0.5 mA*min to about 25 mA*min is administered.

34. A method for the treatment of a fungal infection in a patient suffering therefrom comprising iontophoretically administering the formulation of claim 1 to a body surface of said patient.

35. The method of claim 34 wherein the fungal infection affects the patient's scalp, body, groin, feet, fingernails or toenails.

36. The method of claim 35, wherein the fungal infection is a dermatophyte infection of the nail.

37. The method of claim 36, wherein the dermatophyte infection is onychomycosis.

38. The method of claim 34 wherein the fungal infection is tinea capitis.

39. The method of claim 38 wherein the formulation is administered to the nail plate or to the nail plate and surrounding tissue.

40. A method for the treatment of a fungal infection in a patient suffering therefrom comprising iontophoretically administering an anti-fungal agent to a body surface of said patient.

41. The method of claim 40 wherein the antifungal drug is selected from the group consisting of ketoconazole, econazole, ciclopirox, ciclopirox olamine, terbinafine, fluconazole, itraconazole and amorolfine.
